(19) United States
(12) Patent Application Publication
(10) Pub. No.: US 2020/0346838 A1
(43) Pub. Date: Nov. 5, 2020

(54) DISSOLVABLE PACKAGES OF PRE-MEASURED POWDERED HAIR BLEACH

(71) Applicant: L'Oreal, Paris (FR)

(72) Inventors: Michael Robinson, Brooklyn, NY (US); Jason Fyfe, Homdel, NJ (US)

(73) Assignee: L'Oreal, Paris (FR)

(21) Appl. No.: 16/860,946

(22) Filed: Apr. 28, 2020

Related U.S. Application Data
(60) Provisional application No. 62/840,944, filed on Apr. 30, 2019.

(51) Int. Cl. B65D 81/32; A61K 8/02; A45D 33/00; A45D 34/00; B65D 65/46

(52) U.S. Cl. CPC B65D 81/3261; A61K 8/022; A61K 2800/56; A45D 34/00; B65D 65/46; A45D 33/005

(57) ABSTRACT

Powdered hair bleach individually packaged in pre-measured sizes, weights, or volumes. The powdered hair bleach is encased in a dissolvable substrate. Packages include one or more chambers and include additional compositions.

### CROSS-REFERENCE(S) TO RELATED APPLICATION

This application claims the benefit of U.S. Provisional Application No. 62/840,944, filed Apr. 30, 2019, of which is expressly incorporated herein by reference in its entirety.

### SUMMARY

In one embodiment, a package comprises a water-dissolvable substrate forming the exterior of the package; and a pre-measured powdered hair bleach composition in a chamber of the package.

In one embodiment, a package comprises a water-dissolvable substrate forming the exterior of the package; a pre-measured powdered hair bleach composition in at least one chamber of the package; and a second composition in a second chamber of the package, the second chamber being separated by a barrier from the first chamber, and the second composition being the same or different from the powdered hair bleach composition.

In one embodiment, a package comprises a water-dissolvable substrate forming the exterior of the package; a pre-measured powdered hair bleach composition in at least one chamber of the package; and an additive composition in a second chamber of the package, the second chamber being separated by a barrier from the first chamber, and the additive composition configured to enhance a property of the powdered hair bleach.

In one embodiment, a package comprises a water-dissolvable substrate forming the exterior of the package; a pre-measured powdered hair bleach composition in at least one chamber of the package; and an anhydrous liquid composition in a second chamber of the package, the second chamber being separated by a barrier from the first chamber.

In one embodiment, a package comprises a water-dissolvable substrate forming the exterior of the package; and an anhydrous hair bleach developer liquid composition in a chamber of the package.

In one embodiment, a package comprises a water-dissolvable substrate forming the exterior of the package; a pre-measured powdered hair bleach composition in at least one chamber of the package; and an anhydrous liquid hair bleach composition in a second chamber of the package, the second chamber being separated by a barrier from the first chamber.

In one embodiment, a package comprises a water-dissovable substrate forming the exterior of the package; and a pre-measured composition in at least one chamber of the package, wherein the water-dissolvable substrate includes hydrophilic polymers or a disintegrant.

In one embodiment, a package comprises a water-dissolvable substrate forming the exterior of the package; and an anhydrous hair bleach developer liquid composition in a chamber of the package, wherein the water-dissolvable substrate includes a release mechanism triggered by one of at least moisture and friction.

In one embodiment, a package comprises a water-dissolvable substrate forming the exterior of the package; a pre-measured powdered hair bleach composition in at least one chamber of the package; and an anhydrous liquid composition in a second chamber of the package, the second chamber being separated by a barrier from the first chamber, and the water-dissolvable substrate interacts with an external agent to enhance a property of one or both compositions.

In one embodiment, a package comprises a water-dissolvable substrate forming the exterior of the package; and a pre-measured powdered hair bleach composition in at least one chamber of the package, wherein the water-dissolvable substrate is made from woven or non-woven fibers, wherein the fibers are impregnated with a hair bleach developer composition.

In one embodiment, a package comprises a water-dissolvable substrate forming the exterior of the package; and a composition in a chamber of the package, wherein the water-dissolvable substrate is consumed in a reaction with water.

In one embodiment, a package comprises a first and second water-dissolvable substrate forming the exterior of the package; and a first pre-measured composition in a first chamber formed from the first water-dissolvable substrate; and a second pre-measured composition in a second chamber formed from the second water-dissolvable substrate, wherein the first and second water-dissolvable substrates have different rates of dissolution.

In one embodiment, anyone of the packages comprises synthetic or plant-derived dissolvable substrates and compositions.

In one embodiment, a container comprises a plurality of water-soluble packages, each water-soluble package including a water-dissovable substrate forming the exterior of the package; and a pre-measured powdered hair bleach composition in a chamber of the package.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of a dissolvable package according to one embodiment;

FIG. 2 is a diagrammatical illustration of a plurality of the dissolvable packages of FIG. 1 in a flexible or rigid container;

FIG. 3 is a diagrammatical illustration of a cross section of the dissolvable package of FIG. 1 having a barrier separating two chambers;

FIG. 4 is a diagrammatical illustration of a dissolvable package according to one embodiment;

FIG. 5 is a diagrammatical illustration of a cross section of the dissolvable package of FIG. 4 showing a single chamber.

### DETAILED DESCRIPTION

Current hair bleach formulation is prepared by measuring an amount of hair bleach powder with a scoop out of a bulk bag or a rigid container. The powder is then mixed with liquid developer to a desired consistency in ratios based on desired bleaching result. The conventional process of mixing hair bleach powder is highly inaccurate, unhealthy due to dust inhalation and wasteful.

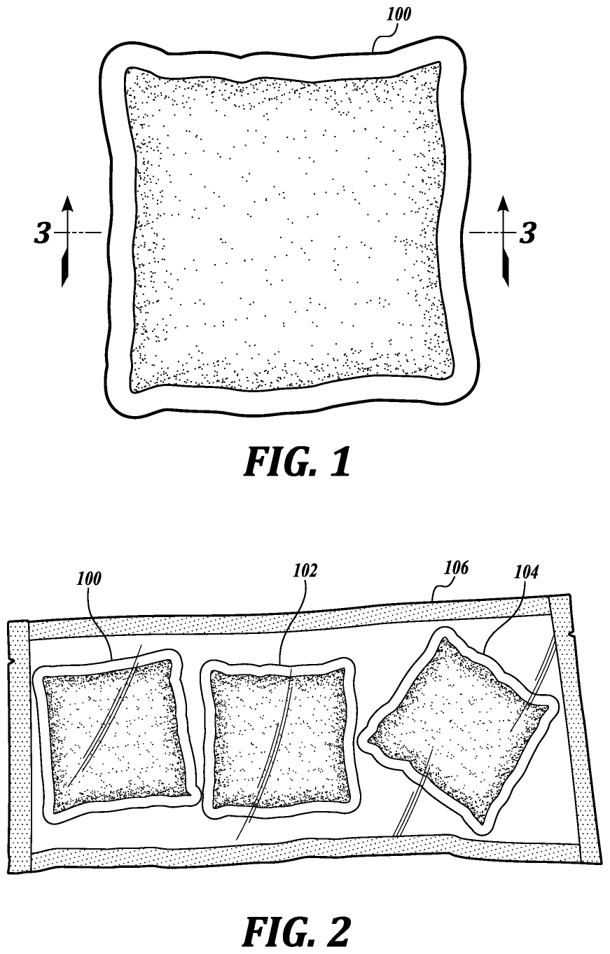

Referring to FIG. 1, a package 100 containing one or more compositions encased in a dissolvable substrate is illustrated. In one embodiment, the compositions can be powder, solid, anhydrous liquid, or a combination. Anhydrous liquid may include any unavoidable moisture that is not sufficient to dissolve the dissolvable substrate from the inside out. The package 100 exterior is made from one or more dissolvable substrates. Specifically, the package 100 exterior will dissolve in aqueous liquids.

In one embodiment wherein the package 100 contains a powdered hair bleach composition, when the package 100 is placed in an aqueous hair bleach developer, for example, the dissolvable substrate will dissolve releasing the hair bleach composition in exact pre-measured weight or volume into the aqueous liquid developer. In one embodiment, the package 100 includes from 1 to 500 grams of any composition disclosed herein. In one embodiment, the package 100 includes from 10 to 50 grams of a hair bleach composition in at least one chamber.

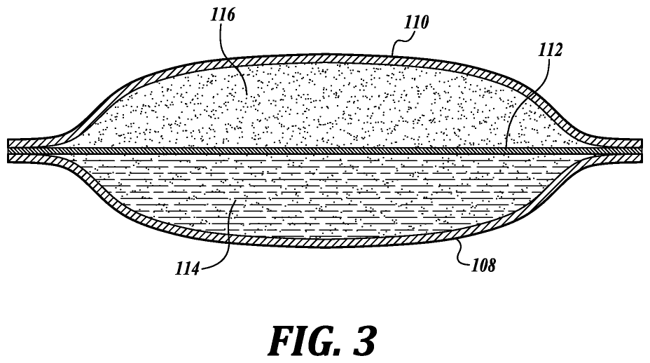

Referring to FIG. 2, individual packages 100, 102, 104 containing the same or different compositions are provided in a flexible or rigid container 106.

Where two compositions are included in the package 100, the compositions can be provided in separate chambers separated by a barrier. Referring to FIG. 3, a cross section of the package 100 is illustrated showing a first 114 and second 116 chamber separated by a barrier 112. Each chamber 114 and 116 can contain the same or a different composition.

FIG. 3 also illustrates one embodiment for making the package 100 from one or more films or sheets 108, 110, 112. In one embodiment, sheets 108, 110, and 114 are the same water-dissolvable composition, and have the same properties, and are the same form. In one embodiment, sheets 108, 110, and 112 are the same water-dissolvable composition, but any one or two sheets might have different properties or be in a different form. For example, sheet 108 can be a water-dissolvable continuous film, while sheet 110 is the same water-dissolvable composition, but made from fibers, such as a woven or non-woven web. In one embodiment, any one or two sheets 108, 110, and 112 is not a water-dissolvable substrate. For example, barrier sheet 112 may be water insoluble. In one embodiment, sheets 108, 110, and 114 are the same composition, and any one or two sheets has a different property. For example, sheet 108 may have a faster dissolution rate than sheet 110 in order to release the composition in chamber 114 before releasing the composition in chamber 116. This is the case where reactions of the compositions need to be in sequential, for example. In one embodiment, any one or more sheets 108, 110, and 114 include multiple layers of the same or different composition.

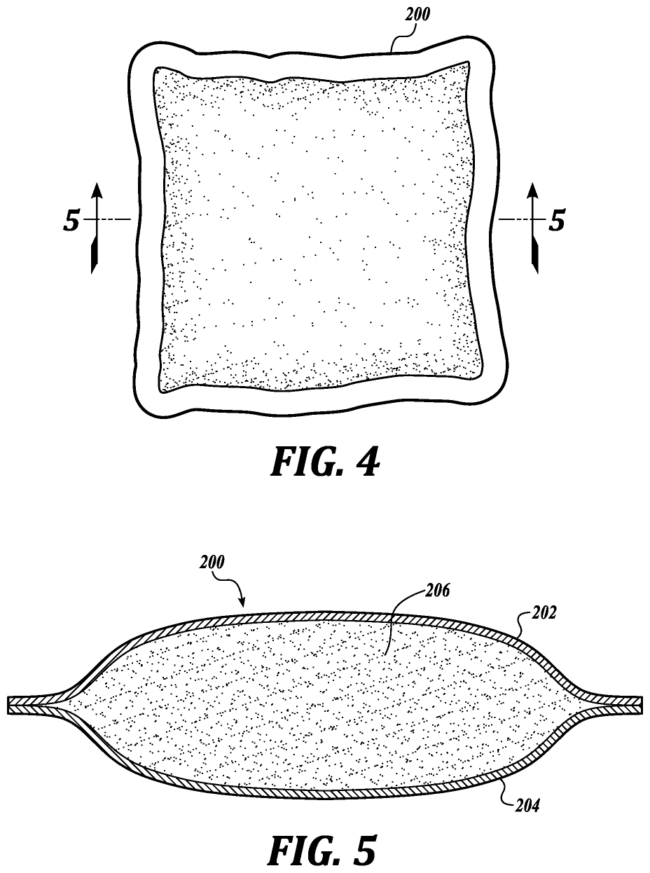

Referring to FIG. 4, a package 200 containing one or more compositions encased in a dissolvable substrate is illustrated. In one embodiment, the compositions can be powder, solid, anhydrous liquid, or a combination. Anhydrous liquid may include any unavoidable moisture that is not sufficient to dissolve the dissolvable substrate from the inside out. The package 200 exterior is made from one or more dissolvable substrates. Specifically, the package 200 exterior will dissolve in aqueous liquids.

In one embodiment wherein the package 200 contains a powdered hair bleach composition, when the package 200 is placed in an aqueous hair bleach developer, for example, the dissolvable substrate will dissolve releasing the hair bleach composition in exact pre-measured weight or volume into the aqueous liquid developer. In one embodiment, the package 200 includes from 1 to 500 grams of any composition disclosed herein. In one embodiment, the package 100 includes from 10 to 50 grams of a hair bleach composition in one chamber.

Referring to FIG. 5, a cross section of the package 200 of FIG. 4 shows a single chamber 206. The chamber 206 can contain a single composition or a mixture of compositions.

FIG. 5 illustrates one embodiment for making the package 200 from one or more films or sheets 202, 204. In one embodiment, sheets 202, 204 are the same water-dissolvable composition, and have the same properties, and are the same form. In one embodiment, sheets 202, 204 are the same water-dissolvable composition, but the two sheets might have different properties or be in a different form. For example, sheet 202 can be a water-dissolvable continuous film, while sheet 204 is the same water-dissolvable composition, but made from fibers, such as a woven or non-woven web. In one embodiment, any one sheet 202, 204 is not a water-dissolvable substrate. In one embodiment, sheets 202, 204 are the same composition, and one sheet has a different property. For example, sheet 202 may have a faster dissolution rate than sheet 204. In one embodiment, one or both sheets 202, 204 include multiple layers of the same or different composition.

In one embodiment, the package 200 without barrier is made from a first 202 and second 204 separate sheets of a dissolvable substrate. For example, a package 200 without barrier can be made by placing sheet 204 of the dissolvable substrate over a plate with perforations connected to a vacuum apparatus, with or without heating depending on flexibility of the dissolvable substrate. As vacuum is applied, the dissolvable substrate 204 is drawn into the perforations to create a depression, which is then filled with a pre-measured weight or volume of a composition. Then, the second sheet 202 of the dissolvable substrate is laid over the first sheet 204 containing the composition. The first 204 and second 202 sheets of dissolvable substrate are then bonded around the periphery to encase the composition. Bonding may include heat welding, adhesives, or creating a bond through chemical reaction. In one embodiment, the second sheet 202 is a flap from the first sheet 204 that has been folded over on itself. Thus, at the one side of the package 100 where the sheet bends onto itself would not need to be bonded.

To add the barrier 112 to form the package 100, the first sheet 108 is placed over a plate with perforations connected to a vacuum apparatus, with or without heating depending on flexibility of the dissolvable substrate. As vacuum is applied, the dissolvable substrate 108 is drawn into the perforations to create a depression, which is then filled with a pre-measured weight or volume of a composition. Then, the second sheet 112 added to the package after vacuuming and filling forms the barrier 112. Then, a second vacuum step depresses the first 108 and second 112 sheets even further creating a second pocket, which is then filled with a pre-measured weight or volume of the second composition. A third sheet 110 of dissolvable substrate is laid over the second composition. Then all three sheets can be bonded and cut at the edges of the package 100. As before, in one embodiment, the third sheet 110 can be a flap from the first sheet 108 that has been folded over on itself, or alternatively, the second sheet 112 can be a flap from the first sheet 108 that has been folded over on itself.

### Embodiment

In accordance with one embodiment, powdered hair bleach is individually packaged in package 200 in pre-measured amounts, encased in a dissolvable substrate 202, 204. In one embodiment, the package 200 includes from 1 to 500 grams of any composition disclosed herein. In one embodiment, the package 200 includes from 20 to 50 grams of any composition disclosed herein. In one embodiment, the package 200 includes from 20 to 50 grams of powdered hair bleach formulas disclosed herein.

### Embodiment

In one embodiment, the package 100 includes two pre-measured powdered hair bleach formulas adjacent to each other in chambers 114, 116, separated by the barrier 106 and incased in a dissolvable substrate 108, 110. In one embodiment, the package 100 includes from 1 to 500 grams of any composition disclosed herein for each chamber 114, 116. In one embodiment, the package 100 includes from 20 to 50 grams of any composition disclosed herein for each chamber 114, 116.

### Embodiment

In one embodiment, the package 100 includes one pre-measured powdered hair bleach formula adjacent to one pre-measured additive formula that provides a secondary benefit in chambers 102, 104, separated by the barrier 106 and incased in a dissolvable substrate 108, 110. In one embodiment, the package 100 includes from 1 to 500 grams of any composition disclosed herein for each chamber 114, 116. In one embodiment, the package 100 includes from 20 to 50 grams of any composition disclosed herein for each chamber 114, 116.

### Embodiment

In one embodiment, the package 100 includes powdered hair bleach formula adjacent to an anhydrous liquid formula in chambers 102, 104, separated by the barrier 106 and incased in a dissolvable substrate 108, 110. In one embodiment, the package 100 includes from 1 to 500 grams of any composition disclosed herein for each chamber 114, 116. In one embodiment, the package 100 includes from 20 to 50 grams of any composition disclosed herein for each chamber 114, 116.

### Embodiment

In one embodiment, the package 200 includes anhydrous hair bleach developer liquid formulation encased in a dissolvable substrate 202, 204. In one embodiment, the package 200 includes from 1 to 500 grams of anhydrous hair bleach developer liquid formulation. In one embodiment, the package 200 includes from 20 to 50 grams of any anhydrous hair bleach developer liquid formulation.

(( ))

### Embodiment

In one embodiment, the package 100 includes anhydrous liquid hair bleach formulation adjacent powdered hair bleach formulation in chambers 114, 116, separated by the barrier 106 and incased in a dissolvable substrate 108, 110. In one embodiment, the package 100 includes from 1 to 500 grams of any composition disclosed herein for each chamber 114, 116. In one embodiment, the package 100 includes from 20 to 50 grams of any composition disclosed herein for each chamber 114, 116.

### Embodiment

In one embodiment, the dissolvable substrates 108, 110, 112, 202, and 204 and the compositions for use in chambers 102, 104, 206 of packages 100, 200 include compositions that are not derived from any animal or include any animal byproducts. In one embodiment, the dissolvable substrates 108, 110, 112, 202, and 204 and the compositions for use in chambers 102, 104, 206 of packages 100, 200 are synthetic, synthetically derived, and/or provided from plants.

### Embodiment

In one embodiment, a plurality of the packages 100 or 200 include hair bleach formulae in individual premeasured amounts encased in dissolvable substrate 108, 110, 112, 202, and 204 and packed into a flexible or rigid container 108.

### Embodiment

In one embodiment, the package 100 or 200 containing dry hair bleach ingredients is made from a rapidly dissolving film substrates 108, 110, 112, 202, and 204, prepared using hydrophilic polymers or a disintegrant.

### Embodiment

In one embodiment, a package 200 includes an anhydrous hair bleach developer liquid formulation encased in a dissolvable substrate 202, 204. In this embodiment, the dissolvable substrates 202, 204 are designed with a secondary release mechanism that is unique to the end-use application. For example, in one embodiment, the dissolvable substrate 202, 204 are more sensitive to dissolution in the presence of moisture and friction, such as from an application brush. In this embodiment, the dissolvable substrate 202, 204 are selected to interact and enhance specific functionality of the product in the presence of an external agent (moisture etc.).

### Embodiment

In one embodiment, a package 100 includes powdered hair bleach formula adjacent to an anhydrous liquid formula, separated by a barrier 112 and both encased in a dissolvable substrate 108, 110. In this embodiment, the dissolvable substrate 108, 110 interacts and enhances specific functionality of the product in presence of an external agent (moisture etc.). In this embodiment, the dissolvable substrate 108, 110 protects and retains efficacy; is shelf stable; can dissolve or disintegrate without residue, and is specific to support the bleach ingredient chemistries.

### Embodiment

In one embodiment, hair bleach formulae in a package 200 in individual pre-measured amounts encased in dissolvable substrate 202, 204 and packed into a flexible or rigid container 106. In this embodiment, the dissolvable substrate 202, 204 serves as a activating tool. In an embodiment, the dissolvable substrate 202, 204 is a woven or non-woven dissolvable substrate with powder impregnation. In one embodiment, the dissolvable substrate 202, 204 is compounded and cured with a powder bleach formulation. In one embodiment, the dissolvable substrate 202, 204 is a non-woven or woven material impregnated with developer formulation. In one embodiment, the dissolvable substrate 202, 204 is compounded with developer formulation.

### Embodiment

In one embodiment, the dissolvable substrate 108, 110, 112, 204, 206 is a rapidly dissolving film containing dry hair bleach ingredients, prepared using hydrophilic polymers. In this embodiment, dissolution rates are enhanced by a sacrificial reaction. In this embodiment, the dissolvable substrate 108, 110, 112, 204, 206 produces little to zero residue, because the dissolvable film is consumed in reaction.

### Embodiment

In one embodiment, the selection of components and how they are put together create unique developer/film/powder dissolution mechanics/chemical kinetics. In this embodiment, compositions and methods of using the unit dose packaging is driven by the end-use application, for example, packages classified by strength, desired hair color etc. In one embodiment, a package 100 includes two chambers 114, 116, each with a pre-measured amount of respective, first and second hair bleach composition. In one embodiment, the dissolution rate for the dissolvable substrates 108, 110 is different, so that the first hair bleach composition is released first, and optionally after adding more developer, the second hair bleach composition is released second.

### Dissolvable Substrates

In one embodiment, the packages 100 and 200 are made from the dissolvable substrates 108, 110, 112, 202, 204 that dissolve in water. In one embodiment, the dissolvable substrate is water soluble when placed in an aqueous composition having at least 5% water by weight. In one embodiment, the dissolvable substrate is water soluble when placed in an aqueous composition having at least 10% water by weight. In one embodiment, the dissolvable substrate is water soluble when placed in an aqueous composition having at least 15% water by weight. In one embodiment, the term “water soluble” means soluble in water, in particular in a proportion of at least 10 grams per liter of water, preferably at least 20 g/l and better still at least 50 g/l, at a temperature of less than or equal to 35 .degree. C. In one embodiment, the term “liposoluble” means soluble in a liquid fatty substance, in particular in a proportion of at least 10 grams per liter of liquid fatty substance, in particular in a plant oil or mineral oil such as liquid petroleum jelly, preferably at least 20 g/l in a liquid fatty substance, better still at least 50 g/l in a fatty substance, at a temperature of less than or equal to 35° C. The term “temperature of less than or equal to 35° C.” is intended to mean a temperature not exceeding 35° C. but greater than or equal to 0° C., for example ranging from more than 1 to 35° C., better still from 5 to 30° C. and even better still from 10 to 30° C. or 10 to 20° C. It is understood that all the temperatures are given at atmospheric pressure.

In one embodiment, the package 100 or 200 is water-soluble or liposoluble at a temperature of less than or equal to 35° C.

In one embodiment, the dissolvable substrates 108, 110, 112, 202, 204 include greater than 0% by weight to 100% by weight poly(vinyl alcohol).

In one embodiment, the dissolvable substrates 108, 110, 112, 202, 204 include greater than 0% by weight to 100% by weight of a polysaccharide.

In one embodiment, the dissolvable substrates 108, 110, 112, 202, 204 include 100% or greater than 0% combined microcrystalline cellulose and maltodextrin.

In one embodiment, the (—OH groups) of poly(vinyl alcohol)(PVA) present in the dissolved mixture act as free radical receptors and for the same volume of hydrogen peroxide reduces the volume of oxygen available for bleaching hair causing reduced lift. In one embodiment, the lift level is compared using higher volumes of hydrogen peroxide. In one embodiment, the poly(vinyl alcohol) concentration is changed, such as by using a thinner or thicker film. In one embodiment, the —OH concentration is reduced by lowering the hydrolysis level of the PVA film.

In one embodiment, PVA in the mixture, when applied to hair and dries, tends to form a film on the hair surface and prevents opening up of some parts of the hair cuticle thereby limiting access to hydrogen peroxide and reducing bleaching levels. In one embodiment, the film forming ability of PVA is prevented by lowering the molecular weight. In one embodiment, more glycerin or polyethylene glycol is added to the developer. In one embodiment, the hydrolysis level of the PVA is adjusted. In one embodiment, a very low molecular weight PVA is used as a plasticizer

In one embodiment, poly(vinyl alcohol) in solution has a slightly acidic pH of 5-6.5 and results in reducing the alkalinity of the bleach-developer mixture. Reduced alkalinity leads to incomplete or limited opening of the hair cuticles and consequently reduced exposure of melanin to the oxidation. In one embodiment, the level of PVA (% vol or wt.) present in the mixture is quantified to establish the likelihood of impact on overall mixture pH. In one embodiment, the pH of the mixture is measured and compared to a control. Then, the pH of the unit dose is adjusted to control levels to evaluate lift. In one embodiment, the hydrolysis level is reduced, thereby reducing the acidity of PVA.

In one embodiment, the dissolvable substrates 108, 110, 112, 202, 204 include greater than 0% by weight to 100% by weight of hydrophillic polymers.

U.S. Pat. No. 10,130,829, incorporated herein by reference, teaches polymers and compositions for use in making the dissolvable substrates 108, 110, 112, 202, 204 for packages 100 and 200.

The polymer(s) contain in their backbones water-soluble units. The water-soluble units are obtained from one or more water-soluble monomers. The term “water-soluble monomer” means a monomer whose solubility in water is greater than or equal to 1% and preferably greater than or equal to 5% at 25 degrees C. and at atmospheric pressure (760 mmHg).

Synthetic water-soluble polymer(s) are advantageously obtained from water-soluble monomers comprising at least one double bond. These monomers may be chosen from cationic, anionic and nonionic monomers, and mixtures thereof. As water-soluble monomers that may be used as precursors of the water-soluble units, alone or as a mixture, examples that may be mentioned include the following monomers, which may be in free or salified form: (meth)acrylic acid, styrenesulfonic acid, vinyl sulfonic acid and (meth)allylsulfonic acid, vinylphosphonic acid, N-vinylacetamide and N-methyl-N-vinylacetamide, N-vinylformamide and N-methyl-N-vinylformamide, N-vinyllactams comprising a cyclic alkyl group containing from 4 to 9 carbon atoms, such as N-vinylpyrrolidone, N-butyrolactam and N-vinylcaprolactam, maleic anhydride, itaconic acid, vinyl alcohol of formula: CH₂═CHOH, vinyl ethers of formula: CH₂═CHOR in which R is a linear or branched, saturated or unsaturated hydrocarbon-based radical containing from 1 to 6 carbons, dimethyldiallylammonium halides (chloride), quaternized dimethylaminoethyl methacrylate (DMAEMA), (meth)acrylamidopropyltrimethylammonium halides (chloride) (APTAC and MAPTAC), methylvinylimidazolium halides (chloride), 2-vinylpyridine and 4-vinylpyridine, acrylonitrile, glycidyl (meth)acrylate, vinyl halides (chloride) and vinylidene chloride, vinyl monomers of formula (I) below:

H₂C═C(R)—C(O)—X (I)

in which formula (I): R is chosen from H and (C₁-C₆)alkyl such as methyl, ethyl and propyl; X is chosen from: alkoxy of —OR′ type in which R′ is a linear or branched, saturated or unsaturated hydrocarbon-based radical containing from 1 to 6 carbon atoms, optionally substituted with at least one halogen atom (iodine, bromine, chlorine or fluorine); a sulfonic (—SO₃⁻), sulfate (—SO₄⁻), phosphate (—PO₄H₂); hydroxyl (—OH); primary amine (—NH₂); secondary amine (—NHR₆), tertiary amine (—NR₆R₇) or quaternary amine (—N±R₆R₇R₈) group with R₆, R₇ and R₈ being, independently of each other, a linear or branched, saturated or unsaturated hydrocarbon-based radical containing 1 to 6 carbon atoms, with the proviso that the sum of the carbon atoms of R′+R₆+R₇+R₈ does not exceed 6; groups —NH₂, —NHR′ and —NR′R″ in which R′ and R″ are, independently of each other, linear or branched, saturated or unsaturated hydrocarbon-based radicals containing 1 to 6 carbon atoms, with the proviso that the total number of carbon atoms of R+R″ does not exceed 6, the said R′ and R″ being optionally substituted with one halogen atom (iodine, bromine, chlorine or fluorine); a hydroxy (—OH); sulfonic (—SO₃⁻), sulfate (—SO₄⁻), phosphate (—PO₄H₂); primary amine (—NH₂); secondary amine (—NHR₆), tertiary amine (—NR₆R₇) and/or quaternary amine (—N⁺R₆R₇R₈) group with R₆, R₇ and R₈ being, independently of each other, a linear or branched, saturated or unsaturated hydrocarbon-based radical containing 1 to 6 carbon atoms, with the proviso that the sum of the carbon atoms of R′+R″+R₆+R₇+R₈ does not exceed 6. As compounds corresponding to this formula, examples that may be mentioned include N,N-dimethylacrylamide and N,N-diethylacrylamide; and mixtures thereof.

Anionic monomers that may especially be mentioned include (meth)acrylic acid, acrylamido-2-methylpropanesulfonic acid, itaconic acid and alkali metal, alkaline-earth metal or ammonium salts thereof or salts thereof derived from an organic amine such as an alkanolamine.

Nonionic monomers that may especially be mentioned include (meth)acrylamide, N-vinylformamide, N-vinylacetamide, hydroxypropyl (meth)acrylate and the vinyl alcohol of formula CH₂═CHOH. The cationic monomers are preferably chosen from quaternary ammonium salts derived from a diallylamine and those corresponding to formula (II) below:

H₂C═C(R₁)-D-N⁺R₂R₃R₄,X⁻ (II)

in which formula (II):

R₁ represents a hydrogen atom or a methyl group,

R₂ and R₃, which may be identical or different, represent a hydrogen atom or a linear or branched C₁-C₄ alkyl group,

R₄ represents a hydrogen atom or a linear or branched C₁-C₄ alkyl group or an aryl group,

D represents the following divalent unit: —(Y)ₙ-(A)-in which: Y represents an amide function, an ester (O—C(O) or C(O)—O), a urethane or a urea, A represents a linear or branched, cyclic or acyclic C₁-C₁₀ alkylene group, which may be substituted or interrupted with a divalent aromatic or heteroaromatic group. The alkylene groups may be interrupted with an oxygen atom, a nitrogen atom, a sulfur atom or a phosphorus atom; the alkylene may be interrupted with a ketone function, an amide, an ester (O—C(O) or C(O) —O), a urethane or a urea, n is an integer ranging from 0 to 1, X⁻ represents an anionic counterion, for instance a chloride or a sulfate.

Examples of water-soluble cationic monomers that may especially be mentioned include the following compounds, and also salts thereof: dimethylaminoethyl, (meth)acryloyloxyethyltrimethylammonium, (meth)acryloyloxyethyldimethylbenzylammonium, N-[dimethylaminopropyl](meth)acrylamide, (meth)acrylamidopropyltrimethylammonium, (meth)acrylamidopropyldimethylbenzylammonium, dimethylaminohydroxypropryl, (meth)acryloyloxyhydroxypropyltrimethylammonium, (meth)acryloyloxyhydroxypropyldimethylbenzylammonium and dimethyldiallylammonium (meth)acrylate.

Preferably, the polymer is polymerized from at least one cationic monomer as defined above. Preferably, the polymers are polymerized from the following monomers comprising at least one double bond as follows: 0 to 30 mol % of acrylic acid, 0 to 95.5 mol % of acrylamide, and 0.5 mol % to 100 mol % of at least one cationic monomer represented in formula (II) as defined above.

As polymers that may be used, mention may be made especially of those polymerized from: 10% of acryloyloxyethyldimethylbenzylammonium chloride and 90% of acrylamide, 30% of acryloyloxytrimethylammonium chloride, 50% of acryloyloxyethyldimethylbenzylammonium chloride and 20% of acrylamide, 10% of acryloyloxyethyltrimethylammonium chloride and 90% of acrylamide, 30% of diallyldimethylammonium chloride and 70% of acrylamide, 30% of acrylic acid and 70% of acrylamide.

According to a particular embodiment, the polymers are polymerized from a cationic monomer and acrylic acid, the number of moles of the cationic monomer being greater than the number of moles of acrylic acid. As water-soluble polymers derived from natural products, mention may be made of polysaccharides, i.e. polymers bearing a sugar unit or sugar units.

The term “sugar unit” means a unit derived from a carbohydrate of formula Cₙ(H₂O)ₙ₋₁ or (CH₂O)ₙ, which may be optionally modified by substitution and/or by oxidation and/or by dehydration. The sugar units that may be included in the composition of the polymers are preferably derived from the following sugars: glucose, galactose, arabinose, rhamnose, mannose, xylose, fucose, fructose, anhydrogalactose, galacturonic acid, glucuronic acid, mannuronic acid, galactose sulfate or anhydrogalactose sulfate.

The polymers bearing a sugar unit or sugar units may be of natural or synthetic origin. They may be nonionic, anionic, amphoteric or cationic. The base units of the polymers bearing a sugar unit may be monosaccharides or disaccharides.

As polymers that may be used, mention may be made especially of the following native gums, and also derivatives thereof:

a) tree or shrub exudates, including: gum arabic (branched polymer of galactose, arabinose, rhamnose and glucuronic acid); ghatti gum (polymer derived from arabinose, galactose, mannose, xylose and glucuronic acid); karaya gum (polymer derived from galacturonic acid, galactose, rhamnose and glucuronic acid); gum tragacanth (or tragacanth) (polymer of galacturonic acid, galactose, fucose, xylose and arabinose);

b) gums derived from algae, including: agar (polymer derived from galactose and anhydrogalactose); alginates (polymers of mannuronic acid and of glucuronic acid); carrageenans and furcellerans (polymers of galactose sulfate and of anhydrogalactose sulfate);

c) gums derived from seeds or tubers, including: guar gum (polymer of mannose and galactose); locust bean gum (polymer of mannose and galactose); fenugreek gum (polymer of mannose and galactose); tamarind gum (polymer of galactose, xylose and glucose); konjac gum (polymer of glucose and mannose) in which the main constituent is glucomannan, a polysaccharide of high molecular weight (500,000<M_{glucomannan}<2,000,000) composed of D-mannose and D-glucose units with a branch every 50 or 60 units approximately;

d) microbial gums, including: xanthan gum (polymer of glucose, mannose acetate, mannose/pyruvic acid and glucuronic acid); gellan gum (polymer of partially acylated glucose, rhamnose and glucuronic acid); scleroglucan gum (glucose polymer); biosaccharide gum (polymer of galacturonic acid, fucose and D-galactose), for example the product sold under the name Fucogel 1.5P from Solabia (polysaccharide rich in fucose (20%) at 1.1% in water and stabilized (1.5% phenoxyethanol));

e) plant extracts, including: cellulose (glucose polymer); starch (glucose polymer); inulin (polymer of fructose and glucose).

These polymers may be physically or chemically modified. A physical treatment that may especially be mentioned is the temperature. Chemical treatments that may be mentioned include esterification, etherification, amidation or oxidation reactions. These treatments can lead to polymers that may be nonionic, anionic, cationic or amphoteric.

Preferably, these chemical or physical treatments are applied to guar gums, locust bean gums, starches and celluloses.

The nonionic guar gums that may be used may be modified with C₁-C₆ hydroxyalkyl groups. Among the hydroxyalkyl groups that may be mentioned are hydroxymethyl, hydroxyethyl, hydroxypropyl and hydroxybutyl groups.

These guar gums are well known in the prior art and may be prepared, for example, by reacting the corresponding alkene oxides, for instance propylene oxides, with the guar gum so as to obtain a guar gum modified with hydroxypropyl groups.

The degree of hydroxyalkylation preferably ranges from 0.4 to 1.2, and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The guar gums modified with cationic groups that may be used more particularly are guar gums comprising trialkylammonium cationic groups. Preferably, 2% to 30% by number of the hydroxyl functions of these guar gums bear trialkylammonium cationic groups. Even more preferentially, 5% to 20% by number of the hydroxyl functions of these guar gums are branched with trialkylammonium cationic groups. Among these trialkylammonium groups, mention may be made most particularly of trimethylammonium and triethylammonium groups. Even more preferentially, these groups represent from 5% to 20% by weight relative to the total weight of the modified guar gum. Use may be made of guar gums modified with 2,3-epoxypropyltrimethylammonium chloride.

These guar gums modified with cationic groups are products already known per se and are, for example, described in patents U.S. Pat. Nos. 3,589,578 and 4,0131,307. Such products are moreover sold especially under the trade names Jaguar C13 S, Jaguar C 15 and Jaguar C 17 by the company Rhodia Chimie.

A modified locust bean gum that may be used is cationic locust bean gum containing hydroxypropyltrimonium groups, such as Catinal CLB 200 sold by the company Toho.

The starch molecules may originate from any plant source of starch, especially cereals and tubers; more particularly, they may be starches from corn, rice, cassava, barley, potato, wheat, sorghum, pea, oat or tapioca. It is also possible to use the starch hydrolysates mentioned above. The starch is preferably derived from potato.

The starches may be chemically or physically modified, especially by one or more of the following reactions: pregelatinization, oxidation, crosslinking, esterification, etherification, amidation and heat treatments.

More particularly, these reactions may be performed in the following manner: pregelatinization by splitting the starch granules (for example drying and cooking in a drying drum);

oxidation with strong oxidizing agents, leading to the introduction of carboxyl groups into the starch molecule and to depolymerization of the starch molecule (for example by treating an aqueous starch solution with sodium hypochlorite);

crosslinking with functional agents capable of reacting with the hydroxyl groups of the starch molecules, which will thus bond together (for example with glyceryl and/or phosphate groups);

esterification in alkaline medium for the grafting of functional groups, especially C₁-C₆ acyl (acetyl), C₁-C₆ hydroxyalkyl (hydroxyethyl or hydroxypropyl), carboxymethyl or octenylsuccinic.

It is possible in particular to obtain, by crosslinking with phosphorus compounds, monostarch phosphates (of the type Am—O—PO—(OX)₂), distarch phosphates (of the type Am—O—PO—(OX)—O—Am) or even tristarch phosphates (of the type Am—O—PO—(O—Am)₂) or mixtures thereof may especially be obtained by crosslinking with phosphorus compounds, Am meaning starch and X especially denoting alkali metals (for example sodium or potassium), alkaline-earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetyl cassava distarch phosphate) by the company Avebe, or Structure Zea from National Starch (gelatinized corn distarch phosphate).

A preferred starch is a starch that has undergone at least one chemical modification such as at least one esterification.

Amphoteric starches comprising one or more anionic groups and one or more cationic groups may also be used. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The amphoteric starches are especially chosen from the compounds having the following formulae:

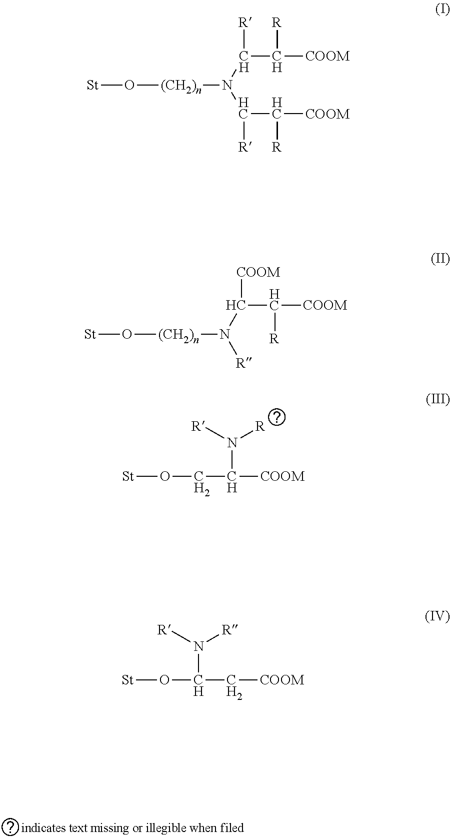

in which formulae (I) to (IV):

St-O represents a starch molecule;

R, which may be identical or different, represents a hydrogen atom or a methyl radical;

R′, which may be identical or different, represents a hydrogen atom, a methyl radical or a group —C(O)—OH;

n is an integer equal to 2 or 3; M, which may be identical or different, denotes a hydrogen atom, an alkali metal or alkaline-earth metal such as Na, K or Li, a quaternary ammonium NH₄, or an organic amine, R″ represents a hydrogen atom or a C₁-C₁₈ alkyl radical.

These compounds are especially described in U.S. Pat. Nos. 5,455,340 and 4,017,460.

Use is particularly made of the starches of formula (II) or (III); and preferentially starches modified with 2-chloroethylaminodipropionic acid, i.e. starches of formula (II) or (III) in which R, R′, R″ and M represent a hydrogen atom and n is equal to 2. The preferred amphoteric starch is a starch chloroethylamidodipropionate.

The celluloses and cellulose derivatives may be anionic, cationic, amphoteric or nonionic.

Among these derivatives, cellulose ethers, cellulose esters and cellulose ester ethers are distinguished.

Among the cellulose esters, mention may be made of mineral cellulose esters (cellulose nitrates, sulfates and phosphates), organic cellulose esters (cellulose monoacetates, triacetates, amidopropionates, acetate butyrates, acetate propionates and acetate trimellitates), and mixed organic/mineral cellulose esters, such as cellulose acetate butyrate sulfates and cellulose acetate propionate sulfates.

Among the cellulose ester ethers, mention may be made of hydroxypropylmethylcellulose phthalates and ethylcellulose sulfates.

Among the nonionic cellulose ethers that may be mentioned are alkylcelluloses such as methylcelluloses and ethylcelluloses (for example Ethocel Standard 100 Premium from Dow Chemical); hydroxyalkylcelluloses such as hydroxymethylcelluloses and hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and also the sodium salts thereof.

Among the cationic cellulose ethers, mention may be made of crosslinked or non-crosslinked, quaternized hydroxyethylcelluloses. The quaternizing agent may especially be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

Among the associative polymers bearing a sugar unit or sugar units, mention may be made of celluloses or derivatives thereof, modified with groups comprising at least one fatty chain, such as alkyl, arylalkyl or alkylaryl groups or mixtures thereof in which the alkyl groups are of C8-C22; nonionic alkylhydroxyethylcelluloses such as the products Natrosol Plus Grade 330 CS and Polysurf 67 (C₁₆ alkyl) sold by the company Aqualon; quaternized alkylhydroxyethylcelluloses (cationic), such as the products Quatrisoft LM 200, Quatrisoft LM-X 529-18-A, Quatrisoft LM-X 529-18-B (C₁₂ alkyl) and Quatrisoft LM-X 529-8 (C₁₈ alkyl) sold by the company Amerchol, the products Crodacel QM and Crodacel QL (C₁₂ alkyl) and Crodacel QS (C₁₈ alkyl) sold by the company Croda, and the product Softcat SL 100 sold by the company Amerchol; nonionic nonoxynylhydroxyethylcelluloses such as the product Amercell HM-1500 sold by the company Amerchol; nonionic alkylcelluloses such as the product Bermocoll EHM 100 sold by the company Berol Nobel.

As associative polymers bearing a sugar unit or sugar units derived from guar, mention may be made of hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

The polymer(s) bearing a sugar unit or sugar units are preferably chosen from guar gums, locust bean gums, xanthan gums, starches and celluloses, in their modified form (derivatives) or unmodified.

Preferably, the polymers bearing a sugar unit or sugar units are nonionic.

More preferably, the polymer(s) bearing a sugar unit or sugar units are chosen from modified nonionic guar gums, especially modified with C₁-C₆ hydroxyalkyl groups.

In one embodiment, dissolvable substrates 108, 110, 112, 202, 204 are made from fibers constituted of one or more water-soluble polymers described herein.

The term “fiber” is intended to mean any object of which the length is greater than its cross section. In other words, it should be understood as meaning an object of length L and of diameter D such that L is greater and preferably very much greater (i.e. at least three times greater) than D, D being the diameter of the circle in which the cross section of the fiber is inscribed. In particular, the ratio L/D (or aspect ratio) is chosen in the range from 3.5 to 2500, preferably from 5 to 500 and better still from 5 to 150. The cross section of a fiber may have any round, toothed or fluted shape, or alternatively a bean shape, but also multilobate, in particular trilobate or pentalobate, X-shaped, ribbon-shaped, square, triangular, elliptical or the like. The fibers may or may not be hollow. The fibers may be of natural, synthetic or even artificial origin. Advantageously, said fibers are of synthetic origin.

Mention may be made more particularly of water-soluble fibers that include fibers based on polyvinyl alcohol (PVA), fibers of polysaccharides such as glucomannans, starches or celluloses such as carboxymethylcelluloses, polyalginic acid fibers, polylactic acid fibers and polyalkylene oxide fibers, and also mixtures thereof. More preferentially, the water-soluble fiber(s) are chosen from PVA-based fibers.

In one embodiment, the dissolvable substrates 108, 110, 112, 202, 204 comprise natural, artificial or synthetic water-soluble polymer fibers, preferably chosen from polyvinyl alcohol (PVA) fibers, polysaccharide fibers such as cellulose and more specifically hydroxyalkylcelluloses, polylactic acid fibers and polyalkylene oxide fibers, and mixtures thereof; more preferably selected from PVA and hydroxyl(C₁-C₆)alkylcelluloses.

A “natural fiber” is a fiber that is present in nature, directly or after mechanical and/or physical treatment. Fibers of animal or plant origin, such as cellulose fibers, in particular extracted from wood, leguminous plants or algae, and rayon fibers, are collated in this category.

The “artificial fibers” are either totally synthetic or derived from natural fibers that have been subjected to one or more chemical treatments in order in particular to improve their mechanical and/or physicochemical properties.

The “synthetic fibers” collate fibers obtained by chemical synthesis and are generally fibers constituted of one or more mono-component or multi-component, composite or non-composite polymers and/or copolymers, which are generally extruded and/or drawn to the desired diameter of the fiber.

The fibers may be spun, carded or twisted. Advantageously, the fibers used are spun. The mean diameter of the fibers which may be identical or different, is less than 500μιη. Advantageously, such a diameter is less than 200μιη, preferably less than 100μιη or even less than 50μπΘ.

In one embodiment, the fibers of the dissolvable substrates 108, 110, 112, 202, 204 are entangled. The packages 100, 200 may be constituted entirely of water-soluble fibers or a sheet which may comprise both water-soluble fibers and fibers that are insoluble in water at a temperature of less than or equal to 35° C., the soluble fibers necessarily being in a larger amount than the insoluble fibers. The sheet of fibers should comprise at least 60% by weight, preferably at least 70% by weight and better still at least 80%> by weight of soluble fibers relative to the total weight of fibers. It may thus comprise, for example, more than 95% by weight, or even more than 99% by weight and even 100% by weight of water-soluble fibers relative to the total weight of fibers in the envelope or the sheets.

When the sheet of fibers contains insoluble fibers, the latter fibers may be made of any material usually used as insoluble fibers; they may be, for example, silk fiber, cotton fiber, wool fiber, flax fiber, polyamide (Nylon®) fiber, polylactic acid fiber, modified cellulo se (rayon, viscose or rayon acetate) fiber, poly-p-phenyleneterephthalamide fiber, in particular Kevlar® fiber, polyolefin fiber and in particular polyethylene or polypropylene fiber, glass fiber, silica fiber, aramid fiber, carbon fiber, in particular in graphite form, Teflon® fiber, insoluble collagen fiber, polyester fiber, polyvinyl or polyvinylidene chloride fiber, polyethylene terephthalate fiber, and fibers formed from a mixture of the compounds mentioned above, for instance polyamide/polyester or viscose/polyester fibers.

In addition, one or more of the dissolvable substrates 108, 110, 112, 202, 204 may be woven or nonwoven.

According to a particular embodiment, one or more of the dissolvable substrates 108, 110, 112, 202, 204 are woven. A “woven” material results from an organized assembly of fibers, in particular of water-soluble polymeric fibers, and more particularly of an intercrossing, in the same plane, of said fibers, arranged in the warp direction and of fibers arranged, perpendicular to the warp fibers, in the weft direction. The binding obtained between these warp and weft fibers is defined by a weave.

Such a woven material results from an operation directed towards assembling the fibers in an organized manner such as weaving per se, but may also result from knitting.

More particularly, the dissolvable substrates 108, 110, 112, 202, 204 comprising the woven polymeric water-soluble fibers that constitute the packages 100, 200 do not comprise any other additional layer superposed thereon.

According to another particularly advantageous mode, the dissolvable substrates 108, 110, 112, 202, 204 are nonwoven.

“Nonwoven” is intended to mean a substrate comprising fibers, in particular water-soluble polymeric fibers, in which substrate the individual fibers are arranged in a disordered manner in a structure in the form of a sheet and which are neither woven nor knitted. The fibers of the nonwoven are generally bonded together, either under the effect of a mechanical action (for example needle punching, air jet, water jet, etc.), or under the effect of a thermal action, or by addition of a binder.

Such a nonwoven is, for example, defined by standard IS O 9092 as a web or a sheet of directionally or randomly orientated fibers, bonded by friction and/or cohesion and/or adhesion, excluding paper and products obtained by weaving, knitting, tufting or stitching incorporating binding yarns or filaments.

A nonwoven differs from a paper by virtue of the length of the fibers used. In paper, the fibers are shorter. However, there are nonwovens based on cellulose fiber, which are manufactured by a wet-laid process and that have short fibers as in paper. The difference between a nonwoven and a paper is generally the absence of hydrogen bonding between the fibers in a nonwoven.

Very preferentially, the fibers are chosen from synthetic fibers such as PVA fibers. In particular, the envelope and sheets are nonwovens, and preferentially made of nonwoven PVA fibers.

To produce the nonwoven water-soluble dissolvable substrates 108, 110, 112, 202, 204, use is preferably made of PVA fibers that are soluble in water at a temperature of less than or equal to 35° C., for instance the fibers sold by the Japanese company Kuraray under the name Kuralon K-II, and particularly the grade WN2 which is soluble at and above 20° C. These fibers are described in document EP-A-636 716 which teaches the manufacture of PVA fibers that are soluble in water at temperatures not exceeding 1 00° C., by spinning and drawing the polyvinyl alcohol polymer in dry or wet form in the presence of solvents participating in the dissolution and solidification of the fiber. The fiber thus obtained may lead to the production of woven or nonwoven substrates.

These fibers may also be prepared from a solution to be spun, by dissolving a water-soluble PVA-based polymer in a first organic solvent, spinning the solution in a second organic solvent to obtain solidified filaments and wet-drawing of the filaments from which the first solvent is removed, and which are then dried and subjected to a heat treatment. The cross section of these fibers may be substantially circular. These fibers have a tensile strength of at least 2.7 g/dtex (3 g/d). Patent application EP-A-0 636 71 6 describes such PVA-based water-soluble fibers and the process for manufacturing them. For example, the fibers may also be formed by extrusion and deposited on a conveyor to form a sheet of fibers which is then consolidated via a standard fiber bonding technique, for instance needle punching, hot-bonding, calendering or air-through bonding, in which technique the water-soluble sheet passes through a tunnel in which hot air is blown, or hydroentanglement directed towards bonding the fibers via the action of fine j ets of water at very high pressure, which cannot be applied to fibers of which the dissolution temperature is too low pressure.

As has been seen previously, dissolvable substrates 108, 110, 112, 202, 204 are not limited to the use of PVA, and use may also be made of fibers made of other water-soluble materials, provided that these materials dissolve in water having the desired temperature, for example the polysaccharide fibers sold under the name Lysorb by the company Lysac Technologies, Inc. or other fibers based on polysaccharide polymers such as glucomannans or starch.

The dissolvable substrates 108, 110, 112, 202, 204 may comprise a mixture of different fibers that are soluble in water at various temperatures (up to 35° C.).

The fibers may be composites, and they may comprise, for example, a core and a sheath not having the same nature, for example formed from different grades of PVA.

According to a particular embodiment, any one of the dissolvable substrates 108, 110, 112, 202, 204 has a nonwoven comprising water-soluble fibers, alone or as a mixture with insoluble fibers as indicated above, with not more than 40% by weight of insoluble fibers relative to the total weight of the fibers constituting the sheet.

In packages 100, the dissolvable substrates 108, 110, 112 may be identical to each other or may be a different composition, different thicknesses, or density. In package 200, the dissolvable substrates 202, 204 may be identical to each other or may be a different composition, different thicknesses, or density.

In one embodiment, the dissolvable substrates 108, 110, 112, 202, 204 may have a basis weight of less than or equal to 60 g/m², or even less than or equal to 50 g/m² and even better still less than or equal to 45 g/m². In one variant, the basis weight of at least one layer may be greater than 60 g/m².

The dissolvable substrates 108, 110, 112, 202, 204 may dissolve in aqueous compostions. The aqueous composition may simply be water. The aqueous composition may optionally comprise at least one polar solvent. Among the polar solvents that may be used in this composition, mention may be made of organic compounds that are liquid at ambient temperature (25° C.) and at least partially water-miscible.

Examples that may be mentioned more particularly include alkanols such as ethyl alcohol, isopropyl alcohol, aromatic alcohols such as benzyl alcohol and phenylethyl alcohol, or polyols or polyol ethers, for instance ethylene glycol monomethyl, monoethyl or monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

More particularly, if one or more solvents are present, their respective content in the aqueous composition ranges from 0.5% to 20% by weight and preferably from 2% to 10% by weight relative to the weight of said aqueous composition.

### Powdered, Solid, and Anhydrous Hair Bleach Compositions

In one embodiment, the powdered hair bleach formulas include potassium persulfate. In one embodiment, the powdered hair bleach formulas include at least one, more than one, or all of the following: potassium persulfate, sodium metasilicate, sodium persulfate, titanium dioxide, silica, kaolin, and EDTA or equivalents thereof.

In one embodiment, the powdered hair bleach formulas are in the solid phase.

In one embodiment, the powdered hair bleach formulas are encased in a dissolvable substrate.

In one embodiment, the package 100 includes only the powdered hair bleach formula. In one embodiment, the package 100 includes from 5 to 100 grams of the powdered hair bleach formula. In one In one embodiment, the package 100 includes from 20 to 50 grams of the powdered hair bleach formula. In one embodiment, the package 100 includes about 30 grams of the powdered hair bleach formula.

WO2018114886, incorporated herein by reference, teaches hair bleach compositions that are usable in the package 100 in one or both of the chambers 102, 104 and in the package 200 in chamber 206.

In one embodiment, an anhydrous solid composition, comprises: (a) one or more oxidation bases, (b) one or more chemical oxidizing agents chosen from alkali metal percarbonates, alkaline-earth metal percarbonates and mixtures thereof, (c) one or more polymers comprising at least one heterocyclic vinyl monomer, and (d) optionally one or more oxidation couplers.

In one embodiment, the oxidation base(s) are chosen from para-phenylenediamines, bis(phenyl)alkylenediamines, para-aminophenols, bis-para-aminophenols, ortho-aminophenols and heterocyclic bases, the addition salts thereof, the solvates thereof, and mixtures thereof, and preferably from para-phenylenediamines, the addition salts thereof, the solvates thereof, and mixtures thereof.

In one embodiment, the total amount of oxidation base(s) ranges from 0. 1% to 30% by weight, preferably from 0.5% to 15% by weight and more preferentially from 1% to 10% by weight relative to the total weight of the anhydrous solid composition.

In one embodiment, the total amount of chemical oxidizing agent(s), chosen from alkali metal percarbonates, alkaline-earth metal percarbonates and mixtures thereof, ranges from 30% to 55% by weight, and preferably from 35% to 50% by weight, relative to the total weight of the anhydrous solid composition.

In one embodiment, the heterocyclic vinyl monomer is chosen from monomers comprising a 4- to 7-membered heterocycle, and comprising from 1 to 4 identical or different intracyclic heteroatoms, which is optionally fused to a benzene ring and/or optionally substituted; the number of intracyclic heteroatoms being less than the number of ring members of the heterocycle.

In one embodiment, the heterocyclic vinyl monomer is chosen from optionally substituted N-vinyl monomers, preferably from N-vinylpyrrolidone, vinylcaprolactam, N-vinylpiperidone, N-vinyl 3-morpholine, N-vinyl-4-oxazolinone, 2-vinylpyridine, 4-vinylpyridine, 2-vinylquinoline, 1-vinylimidazole and 1-vinylcarbazole, which are optionally substituted, and more preferentially the heterocyclic monomer is optionally substituted N-vinylpyrrolidone.

In one embodiment, the polymer comprising at least one heterocyclic vinyl monomer is the crosslinked or non-crosslinked polyvinylpyrrolidone homopolymer.

In one embodiment, the total amount of polymer(s) comprising at least one heterocyclic vinyl monomer ranges from 5% to 70% by weight, preferably from 10% to 60% by weight and more preferentially from 10% to 35% by weight relative to the total weight of the anhydrous solid composition.

In one embodiment, the oxidation coupler(s) are chosen from meta-phenylenediamines, meta-aminophenols, meta-diphenols, naphthalene-based couplers and heterocyclic couplers, the addition salts thereof, the solvates thereof, and mixtures thereof.

In one embodiment, one or more surfactants can be added, the sufactants being chosen from anionic surfactants, amphoteric or zwitterionic surfactants, non-ionic surfactants, cationic surfactants and mixtures thereof, and more preferentially from anionic surfactants and mixtures thereof.

Although bleach compositions can be in various galenic forms, such as powders, granules, pastes or creams, they are generally packaged in liquid form. However, solid compositions bring many advantages compared with liquid compositions. The process for producing them may not require water, thereby making it possible to adopt a more eco-friendly behaviour, and can also be miniaturized.

The formulations in solid form also make it possible to use raw materials that are unstable or have low stability in liquid formulation.

The term “anhydrous composition” is intended to mean a composition comprising a water content of less than 3% by weight, preferably less than 1% by weight, relative to the weight of the composition. Preferably, this water content is less than 0.5% by weight relative to the weight of the composition. More particularly, the water content ranges from 0 to 1% by weight and preferably from 0 to 0.5% by weight relative to the total weight of the composition. Finally, more particularly, it does not comprise water.

The term “solid composition” is intended to mean a composition that can be in powder, paste or particle form (for example spherical particles such as small balls).

The term “powder” is intended to mean a composition in pulverulent form, which is preferably essentially free of dust (or fine particles). In other words, the particle size distribution of the particles is such that the weight ratio of particles less than or equal to 100 micrometres in size (fines content) and preferably less than or equal to 65 micrometres in size (fines content) is advantageously less than or equal to 5%, preferably less than 2% and more particularly less than 1% (particle size evaluated using a Retsch AS 200 Digit particle size analyser; oscillation height: 1 0.25 mm/screening time: 5 minutes). Advantageously, the particle size is between 100μιη and 3 mm and more particularly between 65μιη and 2 mm.

The term “paste” is intended to mean a composition with a viscosity of greater than 5 poises and preferably greater than 1 0 poises, measured at 25° C. and at a shear rate of I s″¹; this viscosity may be determined using a cone-plate rheometer.

The term “particles” is intended to mean small fractionated objects formed from solid particles aggregated together, of variable shapes and sizes. They may have a regular or irregular shape. They may in particular have a spherical shape (such as granules, granular material, balls), a square shape, a rectangular shape, or an elongated shape such as rods. Spherical particles are quite particularly preferred.

The size of the particles can be, in the largest dimension thereof, between 0.01 and 5 mm, preferably between 0. 1 and 2.5 mm, and better still between 0.5 and 2 mm.

The anhydrous solid composition can be in the form of a compressed solid composition, in particular compressed using a manual or mechanical press.

Anhydrous Solid Composition

Oxidation Bases (A)

In one embodiment, an anhydrous solid composition comprises one or more oxidation bases. Preferably, the oxidation bases are chosen especially from heterocyclic bases and benzene-based bases, the addition salts thereof, the solvates thereof, and mixtures thereof.

The oxidation bases that may be used in the composition are chosen especially from para-phenylenediamines, bis(phenyl)alkylene diamines, para-aminophenols, ortho-aminophenols and heterocyclic bases, the addition salts thereof, the solvates thereof, and mixtures thereof.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylene diamine, N, N-dimethyl-para-phenylenediamine, N,N-diethyl-para-phenylenediamine, N,N-dipropyl-para-phenylenediamine, 4-amino-N,N-diethyl-3-methylaniline, N,N-bis(-hydroxyethyl)-para-phenylenediamine, 4-N,N-bis(-hydroxy ethyl) amino-2-methylaniline, 4-N,N-bis(β-hydroxyethyl) amino-2-chloro aniline, 2-β-hydroxy ethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl- 3-methyl-para-phenylenediamine, N-ethyl-N-(-hydroxy ethyl)-para-phenylenediamine, N-(β,γ-dihydroxypropyl)-para-phenylenediamine, N-(4′-amino phenyl)-para-phenylenediamine, N-phenyl-para-phenylenediamine, 2-β-hydroxyethyloxy-para-phenylenediamine, 2\hat{ }-acetylaminoethyloxy-para-phenylenediamine, N-\hat{ }-methoxy ethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine and the corresponding addition salts with an acid.

Among the abovementioned para-phenylenediamines, preference is more particularly given to para-phenylenediamine, para-toluenediamine, 2-isopropyl-para-phenylenediamine, 2-β-hydroxyethyl-para-pheny lene diamine, 2\hat{ }-hydroxyethyloxy-para-phenylenediamine, 2, 6-dimethyl-para-pheny lene diamine, 2,6-diethyl-para-phenylenediamine, 2,3-dimethyl-para-phenylenediamine, N,N-bis(-hydroxy ethyl)-para-phenylenediamine, 2-chloro-para-phenylenediamine and 2-P-acetylaminoethyloxy-para-phenylenediamine and the corresponding addition salts with an acid.

Among the bis(phenyl)alkylenediamines that may be mentioned are for example N,N′-bis(P-hydroxyethyl)-N,N-bis(4′-aminophenyl)-1,3-diaminopropanol, N,N′-bis(P-hydroxyethyl)-N,N-bis(4′-aminophenyl)ethylenediamine, N,N′-bis(4-aminophenyl)tetramethylenediamine, N, N′-bis(-hydroxy ethyl)-N,N′-bis(4-amino phenyl)tetramethylenediamine, N,N′-bis(4-methylaminophenyl)tetramethylenediamine, N, N′-bis (ethyl)-N,N′-bis(4′-amino-3′-methylphenyl)ethylenediamine and 1, 8-bis(2,5-diaminophenoxy)-3,6-dioxaoctane and the corresponding addition salts.

Among the para-aminophenols that are mentioned are for example para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(-hydroxyethylaminomethyl)phenol and 4-amino-2-fluorophenol and the corresponding addition salts with an acid.

Among the ortho-aminophenols that may be mentioned, for example, are 2-aminophenol, 2-amino-5-methylphenol, 2-amino-6-methylphenol and 5-acetamido-2-aminophenol, and the corresponding addition salts.

Among the heterocyclic bases that may be mentioned, for example, are pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are the 3-aminopyrazolo [1,5-a]pyridine oxidation bases or the corresponding addition salts described, for example, in patent application FR 2 801 308. Examples that may be mentioned include pyrazolo[1,5-a]pyrid-3-yl amine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3, 5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-P-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases that are useful are chosen from 3-aminopyrazolo[1,5-a]pyridines and are preferably substituted on carbon atom 2 with:

a) a (di)(Ci-C6)(alkyl)amino group, said alkyl group possibly being substituted with at least one hydroxyl, amino or imidazolium group;

b) an optionally cationic 5- to 7-membered heterocycloalkyl group comprising from 1 to 3 heteroatoms, optionally substituted with one or more (C1-C6)alkyl groups such as a di(C1-C4)alkylpiperazinium group; or

c) a (Ci-C₆)alkoxy group optionally substituted with one or more hydroxyl groups, such as a β-hydroxyalkoxy group, and the corresponding addition salts.

Among the 3-aminopyrazolo[1,5-a]pyridine bases, it will in particular be preferred to use 2[(3-aminopyrazolo[1,5-a]pyridin-2-yl)oxy]ethanol, and/or 4-(3-aminopyrazolo[1,5-a]pyridin-2-yl)-1, 1-dimethylpiperazin-1-ium chloride and/or the corresponding addition salts or solvates thereof.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2733 749 and DE 195 43 988, for instance 4,5-diamino-1-methylpyrazole, 4,5-diamino-1-(P-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-((3-hydroxy ethyl)-3-methylpyrazole, 4,5-diamino-1-ethyl-3-methylpyrazole, 4,5-diamino-1-ethyl-3-(4 ‘-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2’-amino ethyl) amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(-hydroxyethyl)amino-1-methylpyrazole, and the corresponding addition salts. Use may also be made of 4,5-diamino-1-(-methoxyethyl)pyrazole.

A 4,5-diaminopyrazole will preferably be used and even more preferentially 4,5-diamino-1-(-hydroxyethyl)pyrazole and/or a corresponding salt.

The pyrazole derivatives that may also be mentioned include diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxy ethyl)-1,2-dihydropyrazol-3-one, 2-amino-3-(2-hydroxyethyl)amino-6,7-dihydro-1H,5H-pyrazolo[1,2-a]pyrazol-1-one,

2-amino-3-dimethylamino-6,7-dihydro-1H,5H-pyrazolo[1,2-a]pyrazol-1-one, 2,3-diamino-5,6,7,8-tetrahydro-1H,6H-pyridazino[1, 2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazo 1-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Use will preferably be made of 4,5-diamino-1-(P-hydroxyethyl)pyrazole and/or 2,3-diamino-6,7-dihydro-1H,5H-pyrazolo[1,2-a]pyrazol-1-one and/or 2 [(3-aminopyrazolo[1,5-a]pyridin-2-yl)oxy]ethanol and/or 4-(3-aminopyrazolo[1,5-a]pyridin-2-yl)-1, 1-dimethylpiperazin-1-ium chloride and/or the corresponding salts or solvates thereof as heterocyclic bases.

In general, the addition salts of oxidation bases that may be used in the composition are chosen in particular from the addition salts with an acid such as the hydrochlorides, hydrobromides, sulfates, citrates, succinates, tartrates, lactates, tosylates, benzenesulfonates, phosphates and acetates.

Moreover, the solvates of the oxidation bases more particularly represent the hydrates of said bases and/or the combination of said bases with a linear or branched Ci to C₄ alcohol such as methanol, ethanol, isopropanol or n-propanol. Preferably, the solvates are hydrates.

Preferably, the oxidation base(s) are chosen from para-phenylene diamines, bis (phenyl) alky lene diamines, para-aminophenols, bis-para-aminophenols, ortho-aminophenols and heterocyclic bases, the addition salts thereof, the solvates thereof, and mixtures thereof.

More preferentially, the oxidation base(s) are chosen from para-phenylenediamines, the addition salts thereof, the solvates thereof, and mixtures thereof.

The total amount of oxidation base(s) present in the anhydrous solid composition preferably ranges from 0. % to 30% by weight, more preferentially from 0.5% to 15% by weight and better still from 1% to 10% by weight, relative to the total weight of the anhydrous solid composition.

Chemical Oxidizing Agents (B)

The anhydrous solid composition may also comprise one or more chemical oxidizing agents chosen from alkali metal percarbonates, alkaline-earth metal percarbonates and mixtures thereof.

Preferably, the chemical oxidizing agents present in the anhydrous solid composition are anhydrous, and more preferentially anhydrous and solid, that is to say in the form of a powder, of a paste or of particles (such as balls).

Preferably, the chemical oxidizing agent is sodium percarbonate.

The total amount of chemical oxidizing agent(s), chosen from alkali metal percarbonates, alkaline-earth metal percarbonates and mixtures thereof, present in the anhydrous solid composition preferably ranges from 30% to 55% by weight and more preferentially from 35% to 50% by weight, relative to the total weight of the anhydrous solid composition.

The composition may optionally also comprise one or more additional chemical oxidizing agents that are different from the chemical oxidizing agents (b), as defined previously.

The term “chemical oxidizing agent” is intended to mean an oxidizing agent other than atmospheric oxygen.

The additional chemical oxidizing agent(s), optionally present in the anhydrous solid composition, are preferably anhydrous, and more preferentially anhydrous and solid, that is to say in the form of a powder, of a paste or of particles (such as balls).

More particularly, the additional anhydrous chemical oxidizing agent(s) are chosen from (i) peroxygenated salts, for instance persulfates, perborates, peracids and precursors thereof; (ii) alkali metal bromates or ferricyanides; (iii) solid hydrogen peroxide-generating chemical oxidizing agents such as urea peroxide and polymer complexes that can release hydrogen peroxide, especially those comprising a heterocyclic vinyl monomer such as polyvinylpyrrolidone/HhCh complexes, in particular in powder form, which are different from the polymer comprising at least one heterocyclic vinyl monomer (c), as defined below; (iv) oxidases that produce hydrogen peroxide in the presence of a suitable substrate (for example glucose in the case of glucose oxidase or uric acid with uricase); and mixtures thereof.

According to one particular embodiment, the additional chemical oxidizing agent(s) are chosen from complexes of hydrogen peroxide and of polymer containing as monomer at least one heterocyclic vinyl monomer different from the polymer comprising at least one heterocyclic vinyl monomer (c) as defined below.

More particularly, the heterocyclic vinyl monomer is chosen from monomers comprising a 4- to 6-membered heterocycle, optionally fused to a benzene ring and comprising from 1 to 4 identical or different intracyclic heteroatoms; the number of intracyclic heteroatoms being less than the number of ring members of the heterocycle. Preferably, the number of intracyclic heteroatoms is 1 or 2.

More particularly, the heteroatom(s) are chosen from sulfur, oxygen and nitrogen, preferably from nitrogen and oxygen. In accordance with an even more advantageous embodiment, the monomer comprises at least one intracyclic nitrogen atom.

The vinyl heterocycle may optionally be substituted with one or more C₁ to C₄ and preferably C₁ to C₂ alkyl groups.

Preferably, the heterocyclic monomer is chosen from N-vinyl monomers.

Among the monomers that may be envisaged, mention may be made of the following optionally substituted monomers: N-vinylpyrrolidone, vinylcaprolactam, N-vinylpiperidone, N-vinyl-3-morpholine, N-vinyl-4-oxazolinone, 2-vinylpyridine, 4-vinylpyridine, 2-vinylquinoline, 1-vinylimidazole and 1-vinylcarbazole. Preferably, the monomer is optionally substituted N-vinylpyrrolidone.

In accordance with one particularly advantageous embodiment, the polymer is a homopolymer.

However, it is not excluded to use a copolymer. In such a case, the comonomer(s) are chosen from vinyl acetate, (meth)acrylic acids, (meth)acrylamides and C₁ to C₄ alkyl esters of (meth)acrylic acid, which may be substituted or unsubstituted.

The polymer participating in this complex is preferably water-soluble. It may have variable average molecular weights, preferably between 10³ and 3×10⁶ g/mol, and more preferentially between 10³ and 2×10⁶ g/mol. It is also possible to use mixtures of such polymers.

Advantageously, said complex comprises from 10% to 30% by weight, preferably from 13% to 25% by weight and more preferentially from 18% to 22% by weight of hydrogen peroxide relative to the total weight of the complex.

According to an even more advantageous variant, in this complex, the mole ratio between the heterocyclic vinyl monomer(s) and the hydrogen peroxide ranges from 0.5 to 2 and preferably from 0.5 to 1.

This complex is advantageously in the form of a substantially anhydrous powder.

Complexes of this type are especially described in U.S. Pat. Nos. 5,008,106, 5,077,047, EP 832846, EP 714919, DE 4344131 and DE 19545380 and the other polymer complexes described in U.S. Pat. Nos. 5,008,093, 3,376,110 and 5,183,901.

Examples of complexes that may be mentioned include products such as Peroxydone K-30, Peroxydone K-90 and Peroxydone XL-10 and also complexes formed with hydrogen peroxide and one of the following polymers such as Plasdone K-17, Plasdone K-25, Plasdone K-29/32, Plasdone K-90, Polyplasdone INF-10, Polyplasdone XL-10, Polyplasdone XL, Plasdone S-630, Styleze 2000 Terpolymer and the series of Ganex copolymers, sold by the company ISP.

Preferably, the composition can comprise one or more anhydrous solid additional chemical oxidizing agents, which are different from the chemical oxidizing agents (b), chosen from urea peroxide, perborates, persulfates and mixtures thereof.

According to one particularly preferred embodiment, the anhydrous solid composition does not comprise any additional chemical oxidizing agent which is different from the oxidizing agents (b).

Polymers Comprising at Least One Heterocyclic Vinyl Monomer (C)

The anhydrous solid composition may also comprise one or more polymers comprising at least one heterocyclic vinyl monomer.

More particularly, the heterocyclic vinyl monomer is chosen from monomers comprising a 4- to 7-membered heterocycle, and comprising from 1 to 4 identical or different intracyclic heteroatoms, which is optionally fused to a benzene ring and/or optionally substituted; the number of intracyclic heteroatoms being less than the number of ring members of the heterocycle.

Preferably, the number of intracyclic heteroatoms is 1 or 2.

More particularly, the heteroatom(s) are chosen from sulfur, oxygen and nitrogen, and preferably from nitrogen and oxygen.

In accordance with an even more advantageous embodiment, the monomer comprises at least one intracyclic nitrogen atom.

The vinyl heterocycle may optionally be substituted with one or more C₁ to C₄ and preferably C₁ to C₂ alkyl groups.

Preferably, the heterocyclic monomer is chosen from N-vinyl monomers.

Among the heterocyclic vinyl monomers that may be envisaged, mention may advantageously be made of the following optionally substituted monomers: N-vinylpyrrolidone, vinylcaprolactam, N-vinylpiperidone, N-vinyl-3-morpholine, N-vinyl-4-oxazolinone, 2-vinylpyridine, 4-vinylpyridine, 2-vinylquinoline, 1-vinylimidazole and 1-vinylcarbazole. Preferably, the monomer is optionally substituted N-vinylpyrrolidone.

In accordance with one particularly advantageous embodiment, the polymer is a homopolymer.

However, it is not excluded to use a copolymer. The copolymer can comprise at least two distinct heterocyclic vinyl monomers as described previously, or else at least one heterocyclic vinyl monomer, as described previously, and at least one monomer that is different from the heterocyclic vinyl monomers, as described previously.

In the latter case, the comonomer(s) are preferably chosen from vinyl acetate, (meth)acrylic acids, (meth)acrylamides and C₁ to C₄ alkyl esters of (meth)acrylic acid, which may be substituted or unsubstituted.

The polymer comprising at least one heterocyclic vinyl monomer may be crosslinked or non-crosslinked.

The polymer comprising at least one heterocyclic vinyl monomer is preferably water-soluble. It may have variable average molecular weights, preferably between 10³ and 3×10⁶ g/mol, and more preferentially between 10³ and 2×10⁶ g/mol. It is also possible to use mixtures of such polymers.

Preferably, the polymer comprising at least one heterocyclic vinyl monomer is the crosslinked or non-crosslinked vinylpyrrolidone homopolymer.

The total amount of polymer(s) comprising at least one heterocyclic vinyl monomer, present in the anhydrous solid composition, preferably ranges from 5% to 70% by weight, more preferentially from 10% to 60% by weight and better still from 10% to 35% by weight, relative to the total weight of the anhydrous solid composition.

Oxidation Couplers (D)

The anhydrous solid composition can optionally also comprise one or more oxidation couplers.

Among these oxidation couplers, mention may be made in particular of meta-phenylenediamines, meta-aminophenols, meta-diphenols, naphthalene-based couplers and heterocyclic couplers, the addition salts thereof, the solvates thereof, and mixtures thereof.

Examples that may be mentioned include resorcinol, 2-methyl-5-hydroxyethylaminophenol, 2,4-diaminophenoxyethanol, 1,3-dihydroxybenzene, 1,3-dihydroxy-2-methylbenzene, 4-chloro-1,3-dihydroxybenzene, 2,4-diamino-1-(P-hydroxyethyloxy)benzene, 2-amino-4-(P-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, a-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N—(P-hydroxyethyl)amino-3,4-methylenedioxybenzene, 2,6-bis(P-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, 2-methyl-5-aminophenol, 5-N—(P-hydroxyethyl)amino-2-methylphenol, 3-aminophenol (or meta-aminophenol) and 3-amino-2-chloro-6-methylphenol, the corresponding addition salts with an acid and the corresponding mixtures.

Preferably, the coupler(s) are chosen from meta-phenylenediamines, meta-aminophenols, the addition salts thereof, and mixtures thereof, and more preferentially from 2,4-diaminophenoxyethanol, resorcinol, meta-aminophenol, the addition salts thereof, the solvates thereof, and mixtures thereof.

The addition salts of the oxidation couplers optionally present in the composition are chosen especially from the addition salts with an acid, such as the hydrochlorides, hydrobromides, sulfates, citrates, succinates, tartrates, lactates, tosylates, benzenesulfonates, phosphates and acetates, and the addition salts with a base such as sodium hydroxide, potassium hydroxide, ammonia, amines or alkanolamines.

Moreover, the solvates of the oxidation couplers more particularly represent the hydrates of said couplers and/or the combination of said couplers with a linear or branched C i to C₄ alcohol such as methanol, ethanol, isopropanol or n-propanol. Preferably, the solvates are hydrates.

The total amount of oxidation coupler(s), when they are present in the anhydrous solid composition preferably ranges from 0.1% to 25% by weight, more preferentially from 0.5% to 20% by weight and better still from 1% to 19% by weight, relative to the total weight of the anhydrous solid composition.

Surfactants

The anhydrous solid composition can optionally also comprise one or more surfactants, preferably chosen from anionic surfactants, amphoteric or zwitterionic surfactants, non-ionic surfactants, cationic surfactants and mixtures thereof.

The term “surfactant” is intended to mean an agent comprising at least one hydrophilic group and at least one lipophilic group in its structure, and which is preferably capable of reducing the surface tension of water, and comprising in its structure, as optional repeating units, only alkylene oxide units and/or sugar units and/or siloxane units. Preferably, the lipophilic group is a fatty chain comprising from 8 to 30 carbon atoms.

Preferably, the anhydrous solid composition comprises one or more surfactants chosen from anionic surfactants.

The term “anionic surfactant” is intended to mean a surfactant comprising, as ionic or ionizable groups, only anionic groups. These anionic groups are preferably chosen from the groups such as CO₂H, CO₂⁻, SO₃H, SO₃⁻, OSO₃H, OSO₃⁻, O₂PO₂H, O₂PO₂H⁻ and O₂PO₂², the anionic parts comprising a cationic counterion such as those derived from an alkali metal, an alkaline-earth metal, an amine or an ammonium.

As examples of anionic surfactants that may be used in the composition, mention may be made of alkyl sulfates, alkyl ether sulfates, alkylamido ether sulfates, alkylaryl polyether sulfates, monoglyceride sulfates, alkylsulfonates, alkylamidesulfonates, alkylarylsulfonates, a-olefin sulfonates, paraffin sulfonates, alkyl sulfosuccinates, alkyl ether sulfo succinates, alkylamide sulfosuccinates, alkyl sulfoacetates, acylsarcosinates, acylglutamates, alkyl sulfo succinamates, acylisethionates and N-acyltaurates, polyglycoside-polycarboxylic acid and alkyl monoester salts, acyl lactylates, salts of D-galactoside uronic acids, salts of alkyl ether carboxylic acids, salts of alkylaryl ether carboxylic acids, salts of alkylamido ether carboxylic acids; and the corresponding non-salified forms of all these compounds; the alkyl and acyl groups of all these compounds comprising from 6 to 24 carbon atoms and the aryl group denoting a phenyl group.

These compounds may be oxyethylenated and then preferably comprise from 1 to 50 ethylene oxide units.

The salts of C₆ to C₂₄ alkyl monoesters of polyglyco side-polycarboxylic acids may be chosen from C₆ to C₂₄ alkyl polyglycoside-citrates, C₆ to C₂₄ alkyl polyglycoside-tartrates and C₆ to C₂₄ alkyl polyglycoside-sulfo succinates.

When the anionic surfactant(s) are in salt form, they may be chosen from alkali metal salts such as the sodium or potassium salt and preferably the sodium salt, ammonium salts, amine salts and in particular amino alcohol salts or alkaline-earth metal salts such as the magnesium salts.

Examples of amino alcohol salts that may in particular be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)amino methane salts.

Use is preferably made of alkali metal or alkaline-earth metal salts, and in particular sodium or magnesium salts.

Use is preferably made of (C₆-C₂₄)alkyl sulfates and (C₆-C₂₄)alkyl ether sulfates, which are optionally oxyethylenated, comprising from 2 to 50 ethylene oxide units, and mixtures thereof, in particular in the form of alkali metal salts, alkaline-earth metal salts, ammonium salts or amino alcohol salts. More preferentially, the anionic surfactant(s) is (are) chosen from (C₁₀-C₂₀) alkyl sulfates in the form of alkali metal or alkaline-earth metal salts, and in particular sodium lauryl sulfate and sodium cetostearyl sulfate, and mixtures thereof.

Even better still, it is preferred to use sodium lauryl ether sulfate, in particular those containing 2.2 mol of ethylene oxide, more preferentially (C₁₂-C₂₀)alkyl sulfates such as an alkali metal lauryl sulfate such as sodium lauryl sulfate.

Preferably, the anhydrous solid composition comprises one or more surfactants chosen from amphoteric or zwitterionic surfactants.

Stopped. The amphoteric or zwitterionic surfactant(s) are preferably non-silicone, and are in particular derivatives of optionally quaternized aliphatic secondary or tertiary amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group. Mention may be made in particular of (C₈-C₂₀)alkyl betaines, sulfobetaines, (C₈-C₂₀), alkylamido (C₃-C₈) alkyl betaines, and (C₈-C₂₀) alkylamido (C₆-C₈) alkyl sulfobetaines.

Among the amphoteric or zwitterionic surfactants mentioned above, use is preferably made of (C₈-C₂₀)alkyl betaines such as cocobetaine, and (C₈-C₂₀), alkylamido (C₃-C₈), alkyl betaines such as cocamidopropyl betaine, and mixtures thereof. More preferentially, the amphoteric or zwitterionic surfactant(s) is (are) chosen from cocoamidopropylbetaine and cocobetaine, the sodium salt of diethylaminopropyl laurylaminosuccinamate, or mixtures thereof.

Preferably, the anhydrous solid composition comprises one or more surfactants chosen from cationic surfactants.

The cationic surfactant(s) that may be used in the composition comprise, for example, optionally polyoxyalkylenated primary, secondary or tertiary fatty amine salts, quaternary ammonium salts, and mixtures thereof.

Among the cationic surfactants that may be present in the composition, it is more particularly preferred to choose cetyltrimethylammonium, behenyltrimethylammonium and dipalmitoylethylhydroxyethylmethyl-ammonium salts, and mixtures thereof, and more particularly behenyltrimethylammonium chloride, cetyltrimethylammonium chloride, and dipalmitoylethylhydroxyethylammonium methosulfate, and mixtures thereof.

Preferably, the anhydrous solid composition comprises one or more surfactants chosen from non-ionic surfactants.

Examples of non-ionic surfactants that may be used in the composition are described, for example, in the “Handbook of Surfactants” by M. R. Porter, published by Blackie & Son (Glasgow and London), 1991, pp. 116-178.

Examples of non-ionic surfactants that may be mentioned include:
oxyalkylenated (C₈-C₂₄)alkylphenols;
saturated or unsaturated, linear or branched, oxyalkylenated or glycerolated C8 to C30 alcohols;
saturated or unsaturated, linear or branched, oxyalkylenated C8 to C30 amides;
esters of saturated or unsaturated, linear or branched, C8 to C30 acids and of polyethylene glycols;
polyoxyethylenated esters of saturated or unsaturated, linear or branched, C8 to C30 acids and of sorbitan;
esters of fatty acids and of sucrose;
(C8-C30)alkylpolyglycosides, (C8-C30)alkenylpolyglycosides, optionally oxyalkylenated (0 to 10 oxyalkylene units) and comprising 1 to 15 glucose units, (C8-C30)alkylglucoside esters;
saturated or unsaturated oxyethylenated plant oils;
condensates of ethylene oxide and/or of propylene oxide, inter alia, alone or as mixtures;
N-(C8-C30)alkylglucamine and N-(C8-C30)acylmethylglucamine derivatives;
aldobionamides;
amine oxides; and
oxyethylenated and/or oxypropylenated silicones.

The surfactants contain a number of moles of ethylene oxide and/or of propylene oxide ranging advantageously from 1 to 100, more particularly from 2 to 100, preferably from 2 to 50 and more advantageously from 2 to 30. Advantageously, the non-ionic surfactants do not comprise any oxypropylene units.

In accordance with a preferred embodiment, the non-ionic surfactants are chosen from oxyethylenated C8 to C30 alcohols comprising from 1 to 100 mol and more particularly from 2 to 100 mol of ethylene oxide; polyoxyethylenated esters of saturated or unsaturated, linear or branched C8 to C30 acids and of sorbitan comprising from 1 to 100 mol and better still from 2 to 100 mol of ethylene oxide.

As examples of monoglycerolated or polyglycerolated non-ionic surfactants, monoglycerolated or polyglycerolated C8 to C40 alcohols are preferably used.

In particular, the monoglycerolated or polyglycerolated C8 to C40 alcohols preferably correspond to formula (A8) below:

R₂O—[CH₂—CH(CH₂OH)—O]ₘ—H (A8)

in which:

R₂ represents a linear or branched C8 to C40 and preferably C8 to C30 alkyl or alkenyl radical; and

m represents a number ranging from 1 to 30 and preferably from 1 to 10.

As examples of compounds of formula (A8) that are suitable for use, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol comprising 2 mol of glycerol, cetearyl alcohol comprising 6 mol of glycerol, oleocetyl alcohol comprising 6 mol of glycerol and octadecanol comprising 6 mol of glycerol.

The alcohol of formula (A8) may represent a mixture of alcohols in the same way that the value of m represents a statistical value, which means that, in a commercial product, several species of polyglycerolated fatty alcohols may coexist in the form of a mixture.

Among the monoglycerolated or polyglycerolated alcohols, it is more particularly preferred to use the C₈ to C₁₀ alcohol containing 1 mol of glycerol, the C₁₀ to C₁₂ alcohol containing 1 mol of glycerol and the C12 alcohol containing 1.5 mol of glycerol.

Preferentially, the non-ionic surfactant in the composition is a monooxyalkylenated or polyoxyalkylenated, particularly monooxyethylenated or polyoxyethylenated, or monooxypropylenated or polyoxypropylenated, non-ionic surfactant, or a combination thereof, more particularly monooxyethylenated or polyoxyethylenated, monoglycerolated or polyglycerolated surfactants and alkylpolyglucosides.

More preferably still, the non-ionic surfactants are chosen from polyoxyethylenated sorbitan esters, polyoxyethylenated fatty alcohols and alkylpolyglucosides, and mixtures thereof.

More preferentially, the anhydrous solid composition comprises one or more surfactants chosen from anionic surfactants, non-ionic surfactants and mixtures thereof, and more preferentially from anionic surfactants and mixtures thereof.

Alkaline Agents

The anhydrous solid composition may optionally also comprise one or more alkaline agents.

The alkaline agent(s) can be chosen from silicates and metasilicates such as alkali metal metasilicates, carbonates or hydrogen carbonates of alkali metals or alkaline-earth metals, such as lithium, sodium, potassium, magnesium, calcium or barium, and mixtures thereof.

The alkaline agent(s) can also be chosen from ammonium salts, and in particular inorganic ammonium salts.

Preferably, the ammonium salt(s) are chosen from ammonium halides, such as ammonium chloride, ammonium sulfate, ammonium phosphate, ammonium nitrate and mixtures thereof.

More preferentially, the ammonium salt is ammonium chloride or ammonium sulfate.

In one preferred embodiment, the anhydrous solid composition comprises one or more alkaline agents.

Even more preferentially, the anhydrous solid composition comprises one or more ammonium salts, preferably chosen from ammonium chloride or ammonium sulfate, better still ammonium sulfate.

Thickening Polymers

The anhydrous solid composition may optionally also comprise one or more thickening polymers.

Advantageously, the thickening polymer(s) are chosen from the following polymers:

(a) non-ionic amphiphilic polymers comprising at least one fatty chain and at least one hydrophilic unit;

(b) anionic amphiphilic polymers comprising at least one hydrophilic unit and at least one fatty-chain unit;

(c) crosslinked acrylic acid homopolymers;

(d) crosslinked homopolymers of 2-acrylamido-2-methylpropanesulfonic acid, and crosslinked acrylamide copolymers thereof which are partially or totally neutralized;

(e) ammonium acrylate homopolymers or copolymers of ammonium acrylate and of acrylamide;

(f) dimethylaminoethyl methacrylate homopolymers quatemized with methyl chloride or dimethylaminoethyl methacrylate copolymers quatemized with methyl chloride and acrylamide; and

(g) polysaccharides such as:

(g 1) scleroglucan gums (biopolysaccharide of microbial origin); (g2) gums derived from plant exudates, such as gum arabic, ghatti gum, karaya gum or gum tragacanth;

(g3) celluloses and derivatives;

(g4) guar gums and derivatives; or

(g5) starches or derivatives.

Amphiphilic polymers are more particularly hydrophilic polymers that are capable, in the medium of the composition, and more particularly in an aqueous medium, of reversibly combining with each other or with other molecules.

Their chemical structure more particularly comprises at least one hydrophilic group and at least one hydrophobic group. The term “hydrophobic group” is intended to mean a radical or polymer bearing a saturated or unsaturated, linear or branched hydrocarbon-based chain, comprising at least 8 carbon atoms, preferably at least 10 carbon atoms, more preferentially from 10 to 30 carbon atoms, in particular from 12 to 30 carbon atoms and even better still from 18 to 30 carbon atoms. Preferably, the hydrocarbon-based group is derived from a mono functional compound. By way of example, the hydrophobic group may be derived from a fatty alcohol such as stearyl alcohol, dodecyl alcohol or decyl alcohol. It may also denote a hydrocarbon-based polymer, such as, for example, polybutadiene.

Metabisulfites

The anhydrous solid may optionally also comprise one or more metabisulfites.

The metabisulfite(s) can preferably be chosen from alkali metal or alkaline-earth metal metabisulfites and mixtures thereof, more preferentially from alkali metal metabisulfites and mixtures thereof, and better still from sodium or potassium metabisulfites and mixtures thereof.

Particularly preferably, the metabisulfite is sodium metabisulfite.

The total amount of metabisulfite(s), when they are present in the anhydrous solid composition preferably ranges from 0. 1% to 30% by weight, relative to the total weight of the anhydrous solid composition.

More particularly, the total amount of metabisulfite(s) may range from 0.2% to 20% by weight, more preferentially from 1% to 20%) by weight and better still from 3% to 10%> by weight, relative to the total weight of the anhydrous solid composition.

More particularly, when the metabisulfite is sodium metabisulfite, the total amount of sodium metabisulfite present in the anhydrous solid composition preferably ranges from 0. 1% to 30% by weight, relative to the total weight of the anhydrous solid composition.

More preferentially when the metabisulfite is sodium metabisulfite, the total amount of sodium metabisulfite present in the anhydrous solid composition may range from 0.2% to 20% by weight, better still from 1% to 20% by weight and still from 3% to 10% by weight, relative to the total weight of the anhydrous solid composition.

Additives

The anhydrous solid composition may also optionally comprise one or more additives, different from the compounds and among which mention may be made of cationic, anionic, non-ionic or amphoteric polymers or mixtures thereof, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, in particular polymeric thickeners, opacifiers or nacreous agents, antioxidants, hydroxy acids, fragrances, preservatives, pigments and ceramides.

Needless to say, those skilled in the art will take care to select this or these optional additional compound(s) such that the advantageous properties intrinsically associated with the anhydrous solid composition are not, or are not substantially, adversely affected by the envisaged addition(s).

The above additives may generally be present in an amount, for each of them, of between 0 and 20% by weight relative to the total weight of the anhydrous solid composition.

Cosmetically Acceptable Medium

The term “cosmetically acceptable medium” is intended to meana medium that is compatible with keratin fibers, in particular human keratin fibers such as the hair.

The cosmetically acceptable medium is constituted of water or of a mixture of water and of one or more organic solvents.

Examples of organic solvents that may be mentioned include linear or branched C₂ to C₄ alkanols, such as ethanol, isopropanol, tert-butanol or n-butanol; glycerol; polyols and polyol ethers, for instance 2-butoxyethanol, propylene glycol, hexylene glycol, dipropylene glycol, propylene glycol monomethyl ether, diethylene glycol monomethyl ether and monoethyl ether, and also aromatic alcohols or ethers, for instance benzyl alcohol or phenoxyethanol, and mixtures thereof.

WO2014029657, incorporated herein by reference, teaches hair bleach compositions that are usable in the packages 100, 200 in one or both of the chambers 102, 104, 206.

In one embodiment, a composition for bleaching keratin fibers, comprises at least one persulfate and at least one hydrogen peroxide generator.

In one embodiment, the persulfate(s) is (are) chosen from sodium persulfates, potassium persulfates and ammonium persulfates, and mixtures thereof.

In one embodiment, the persulfate concentration is between 10% and 80% by weight, preferably between 20% and 70% by weight and better still between 40% and 65% by weight relative to the total weight of the composition.

In one embodiment, the hydrogen peroxide generator is chosen from: polymeric complexes that can release hydrogen peroxide, such as polyvinylpyrrolidone/H₂0₂, urea peroxide, alkali metal, alkaline-earth metal or ammonium perborates and percarbonates, in particular the percarbonates, and mixtures thereof.

In one embodiment, the hydrogen peroxide generator is chosen from alkali metal or alkaline-earth metal percarbonates, in particular sodium percarbonate.

In one embodiment, the hydrogen peroxide-generating agent(s) represent(s) from 0.1% to 40% by weight, preferably from 0.5% to 20% by weight and better still from 1% to 10% by weight relative to the total weight of the composition.

In one embodiment, at least one alkaline agent is added, the at least one alkaline agent being chosen from water-soluble silicates such as alkali metal or alkaline-earth metal silicates, dibasic or tribasic alkali metal or alkaline-earth metal phosphates, and alkali metal or alkaline-earth metal carbonates, and mixtures thereof.

In one embodiment, the alkaline agent(s) is (are) present in an amount ranging from 0.1% to 40%, preferably from 0.5% to 30% by weight and better still from 1% to 20% by weight relative to the total weight of the composition.

In one embodiment, at least one rheology modifier is added, the at least one rheology modifier is chosen from hydrophilic thickeners, amphiphilic polymers comprising at least one hydrophobic chain, and fillers, and mixtures thereof.

In one embodiment, at least one disintegration agent is added, the at least one disintegration agent is chosen from celluloses and cellulose derivatives, crosslinked polyacrylates, crosslinked polyvinylpyrrolidone, soybean polysaccharides, alginates, aluminium silicates and derivatives thereof, and hydrophilic silicas, and mixtures thereof.

In one embodiment, the composition comprises an organic inert phase, which is preferably liquid, preferably chosen from the group formed by the polydecenes of formula CionH_{[(20n)+2]} in which n ranges from 3 to 9 and preferably from 3 to 7, and esters of fatty alcohols or of fatty acids, and mixtures thereof.

Persulfates

In one embodiment, compositions include one or more persulfate(s) chosen from sodium persulfates, potassium persulfates and ammonium persulfates, and mixtures thereof.

The persulfate concentration in the composition in is generally between 10% and 80% by weight, preferably between 20% and 70% by weight and better still between 40% and 65% by weight relative to the total weight of the composition.

Alkaline Agents

The alkaline agent(s) may be chosen, for example, from water-soluble silicates such as alkali metal or alkaline-earth metal silicates, such as the dibasic or tribasic ammonium phosphate, sodium disilicate, sodium metasilicate, dibasic or tribasic alkali metal or alkaline-earth metal phosphates or carbonates of alkali metals or alkaline-earth metals, such as lithium, sodium, potassium, magnesium, calcium and barium, and mixtures thereof. Preferably, the alkaline agent(s) are chosen from water-soluble silicates such as alkali metal or alkaline-earth metal silicates, dibasic or tribasic alkali metal or alkaline-earth metal phosphates, and alkali metal or alkaline-earth metal carbonates, and mixtures thereof.

The term “water-soluble silicate” is understood to mean a silicate which has a solubility in water of greater than 0.5% and preferably greater than 1% by weight at 25° C. These water-soluble silicates differ from aluminium silicates and derivatives thereof, in particular clays, such as mixed silicates of natural or synthetic origin that are insoluble in water.

When they are present in the composition, the concentration of alkaline agents generally ranges from 0.1% to 40% by weight, preferably from 0.5% to 30% by weight and better still from 1% to 25% by weight relative to the total weight of the composition.

Hydrogen Peroxide-Generating Agent

As hydrogen peroxide-generating agent may include polymeric complexes that can release hydrogen peroxide, such as polyvinylpyrrolidone/H₂02 in particular in the form of powders, and the other polymeric complexes described in the documents U.S. Pat. Nos. 5,008,093; 3,376,110; 5,183,901, including, but not limited to: urea peroxide, and alkali metal, alkaline-earth metal or ammonium perborates and percarbonates, in particular percarbonates such as sodium percarbonate, and mixtures thereof.

A hydrogen peroxide generator chosen from urea peroxide and alkali metal or alkaline-earth metal percarbonates, in particular sodium percarbonate, is preferably used.

It may be noted that alkali metal, alkaline-earth metal or ammonium persulfates are not included in these precursors since, in the redox mechanisms using these persulfates, there is no release of hydrogen peroxide.

The hydrogen peroxide-generating agent(s) may represent from 1% to 40% by weight, preferably from 5% to 30% by weight and better still from 10% to 20% by weight relative to the total weight of the composition.

Rheology Modifiers

According to one embodiment, the bleaching composition comprises at least one rheology modifier chosen from hydrophilic thickeners, amphiphilic polymers comprising at least one hydrophobic chain, and fillers, and mixtures thereof.

The rheology modifier(s) may be present in a content ranging from 0.01% to 30% by weight, relative to the total weight of the composition, and preferably from 0.1% to 10% by weight.

As examples of hydrophilic thickeners, i.e. thickeners not comprising a C₆-C₃₀ hydrocarbon-based fatty chain, which may be used, mention may be made in particular of:

thickening polymers of natural origin such as

a) algal extracts, such as alginates (for instance alginic acid and sodium alginates), carrageenans and agar agars, and mixtures thereof. Examples of carrageenans that may be mentioned include Satiagum UTC30® and UTC10® from the company Degussa; an alginate that may be mentioned is the sodium alginate sold under the name Kelcosol® by the company ISP;

b) gums, such as xanthan gum, guar gum and non-ionic derivatives thereof (hydroxypropyl guar), gum arabic, konjac gum or mannan gum, gum tragacanth, ghatti gum, karaya gum or locust bean gum; agar gum, and scleroglucan gums, and mixtures thereof;

c) starches, preferably modified starches, such as those derived, for example, from cereals such as wheat, corn or rice, from legumes such as yellow peas, from tubers such as potatoes or manioc, and tapioca starches; carboxymethylstarch. Examples of starches that may be mentioned include the corn starch Starx 15003 sold by the company

Staley, the pregelatinized starch sold under the name Lycatab PGS by the company Roquette; the sodium carboxymethylstarch sold under the reference Explotab by the company Roquette;

d) dextrins, such as dextrin extracted from corn;

e) celluloses such as microcristalline cellulose, amorphous cellulose, and cellulose derivatives, in particular hydroxy(C1-C₆)alkylcelluloses and carboxy(Ci-C₆)alkylcelluloses, which are in particular crosslinked; mention may in particular be made of methylcelluloses, hydroxyalkylcelluloses, ethylhydroxyethylcelluloses and carboxymethylcelluloses. As examples, mention may be made of the microcrystalline cellulose sold under the name Avicel PH 100 or PH102 by the company FMC Biopolymers;

f) pectins;

g) chitosan and derivatives thereof;

h) anionic polysaccharides other than starch and cellulose derivatives, in particular of biotechnological origin, such as anionic polysaccharide bearing as repeating unit a tetrasaccharide composed of L-fucose, D-glucose and glucuronic acid, such as the product bearing the INCI name Biosaccharide Gum-4 sold under the reference Glycofilm 1.5P by the company Solabia;

i) soybean polysaccharides,

and mixtures thereof;

synthetic polymers, such as crosslinked or non-crosslinked polyvinylpyrrolidone, for instance crosslinked polyvinylpyrrolidone, for instance Kollindon CL sold by the company BASF, acrylic acid polymers and salts thereof, for instance crosslinked polyacrylates, such as the product sold by the company Rohm and Haas under the reference Acusol 772, polyacrylamides, crosslinked or non-crosslinked poly(2-acrylamidopropanesulfonic acid) polymers (in particular homopolymers), for instance non-crosslinked poly(2-acrylamidopropanesulfonic acid) (Simugel® EG from the company Seppic), crosslinked poly(2-acrylamido-2-methylpropanesulfonic acid), which is free or partially neutralized with aqueous ammonia (Hostacerin® AMPS from the company Clariant), blends of non-crosslinked poly(2-acrylamido-2-methylpropanesulfonic acid) with hydroxyalkylcellulose ethers or with poly(ethylene oxide)s, as described in U.S. Pat. No. 4,540,510; blends of poly(meth)acrylamido(C₁-C₄ alkyl)sulfonic acid, which is preferably crosslinked, with a crosslinked copolymer of maleic anhydride and of a (C₁ to C₆)alkyl vinyl ether (Stabileze QM from the company ISF).

The amount of hydrophilic thickeners may be between 0.01% and 30% and preferably between 0.1% and 15% by weight and better still between 0.1% and 10% by weight relative to the total weight of the composition.

The compositions may comprise at least one amphiphilic polymer comprising at least one hydrophobic chain.

More especially, if they are present, these amphiphilic polymers are of non-ionic, anionic, cationic or amphoteric type. They are preferably of non-ionic, anionic or cationic nature.

Said amphiphilic polymers comprise, more particularly, as hydrophobic chain, a saturated or unsaturated, aromatic or non-aromatic, linear or branched C₆-C₃₀ hydrocarbon-based fatty chain, attached to optionally one or more oxyalkylene (oxyethylene and/or oxypropylene) units.

Among the cationic amphiphilic polymers comprising a hydrophobic chain are cationic polyurethanes or cationic copolymers comprising vinyllactam and in particular vinylpyrrolidone units.

Even more preferentially, the amphiphilic polymers comprising a hydrophobic chain are of non-ionic or anionic nature.

Examples of hydrophobic-chain non-ionic amphiphilic polymers that may be mentioned, inter alia, include celluloses comprising a hydrophobic chain (Natrosol Plus Grade 330 CS® from the company Aqualon; Bermocoll EHM 100® from the company Berol Nobel; Amercell Polymer HM-1500® from the company Amerchol); hydroxypropyl guars modified with one or more hydrophobic groups (Jaguar XC-95/3®, RE210-18, RE205-1 from the company Rhodia Chimie; Esaflor HM 22® from the company Lamberti); copolymers of vinylpyrrolidone and of hydrophobic-chain monomers (certain products of the Antaron® and Ganex® ranges from the company ISP); copolymers of C₁ to C₆ alkyl (meth)acrylates and of amphiphilic monomers comprising a hydrophobic chain; copolymers of hydrophilic (meth)acrylates and of monomers comprising at least one hydrophobic chain (polyethylene glycol methacrylate/lauryl methacrylate copolymer); polymers with an aminoplast ether backbone containing at least one fatty chain (Pure Thix® from the company Sud-Chemie); polyether polyurethanes, of linear (block structure), grafted or star form, comprising in their chain at least one hydrophilic block and at least one hydrophobic block (as described in the article by G. Fonnum, J. Bakke and Fk. Hansen—Colloid Polym. Sci 271, 380.389 (1993J; in particular the polyether polyurethane which can be obtained by polycondensation of at least three compounds comprising (i) at least one polyethylene glycol comprising from 150 to 180 mol of ethylene oxide, (ii) a polyoxyethylented stearyl alcohol comprising 100 mol of ethylene oxide and (iii) a diisocyanate, as sold in particular by the company Elementis under the name Rheolate FX 1 100®, which is a polycondensate of polyethylene glycol comprising 136 mol of ethylene oxide, of polyoxyethylented stearyl alcohol comprising 100 mol of ethylene oxide and of hexamethylene diisocyanate (HDI) having a weight-average molecular weight of 30 000 (INCI name: PEG-136/Steareth-100/SMDI Copolymer). Mention may also be made of Rheolate® 205, 208, 204 or 212 from the company Rheox; Elfacos® T210, T212 from the company Akzo).

As examples of anionic amphiphilic polymers comprising at least one hydrophobic chain that may be used, mention may be made of crosslinked or non-crosslinked polymers comprising at least one hydrophilic unit derived from one or more ethylenically unsaturated monomers comprising a carboxylic acid function, which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Mention may be made in particular of copolymers of (meth)acrylic acid and of C₁₀-C₃₀ alkyl (meth)acrylates, which are crosslinked or non-crosslinked, such as those described in U.S. Pat. Nos. 3,915,921 and 4,509,949, or copolymers of (meth)acrylic acid and of fatty alcohol allyl ethers such as those described in EP 216 479.

In addition, the products Carbopol ETD-2020® and 1382®, Pemulen TR10 and TR20 from the company Goodrich; the methacrylic acid/ethyl acrylate/oxyethylenated stearyl methacrylate copolymer (55/35/10); the (meth)acrylic acid/ethyl acrylate/oxyethylenated behenyl methacrylate 25 EO copolymer; the methacrylic acid/ethyl acrylate/steareth-10 allyl ether crosslinked copolymer, are polymers that are suitable for use.

If these amphiphilic polymers are present, their content represents from 0.01% to 30% by weight and preferably from 0.1% to 10% by weight relative to the weight of the composition.

“Fillers” should be understood as meaning solid particles which are insoluble in the medium of the composition, whatever the temperature at which the composition is manufactured.

The fillers may be colourless and inorganic or organic, of any physical shape (platelet, spherical or oblong) and of any crystallographic form (for example sheet, cubic, hexagonal, orthorhombic, etc.). The fillers may be porous or non-porous.

Fillers that may be mentioned include inorganic fillers such as hydrophobic or hydrophilic silicas, clays other than those mentioned above, ceramic beads, magnesium oxides, aluminium silicates and derivatives thereof, in particular clays, such as mixed silicates of natural or synthetic origin, in particular magnesium aluminium silicates, which are in particular hydrated, natural hydrated aluminium silicates, such as bentonite or kaolin, talc, organic fillers such as Nylon, microspheres based on a copolymer of vinylidene chloride/acrylonitrile/methacrylonitrile containing isobutane, and expanded, such as those sold under the name Expancel 551 DE® by the company Expancel, micronized plant powder (such as the fruit powders from the company Lessonia) or non-micronized plant powder, or alternatively rice grain husk powder, and mixtures thereof.

Among the silicas, mention may also be made in particular of fumed silicas of hydrophilic nature (in particular Aerosil® 90, 130, 150, 200, 300 and 380 from the company Degussa Huls).

Some of the rheology modifiers mentioned above may also play a role in aiding the disintegration of the bleaching composition in compressed form during its use.

Thus, in one particular embodiment, the composition comprises at least one disintegration agent chosen from celluloses, in particular microcrystalline cellulose, and cellulose derivatives, crosslinked polyacrylates, crosslinked polyvinylpyrrolidone, soybean polysaccharides, alginates, aluminium silicates and derivatives thereof, and silicas, in particular hydrophilic silicas, and mixtures thereof.

Surfactants

The compositions may advantageously comprise at least one surfactant.

The surfactant(s) may be chosen indiscriminantly, alone or as mixtures, from anionic, amphoteric, non-ionic, zwitterionic and cationic surfactants, in particular from anionic and/or non-ionic surfactants.

Among the non-ionic surfactants, mention may be made of alcohols, alpha-diols and alkyl phenols, each of these compounds being polyethoxylated and/or polypropoxylated, and containing at least one hydrocarbon-based chain comprising, for example, from 8 to 30 carbon atoms and preferably from 8 to 18 carbon atoms, the number of ethylene oxide and/or propylene oxide groups possibly ranging in particular from 2 to 200.

Mention may also be made of copolymers of ethylene oxide and propylene oxide, condensates of ethylene oxide and/or of propylene oxide with fatty alcohols; polyethoxylated fatty amides preferably having from 2 to 30 mol of ethylene oxide; oxyethylenated fatty acid esters of sorbitan containing from 2 to 30 mol of ethylene oxide; fatty acid esters of sucrose, fatty acid esters of polyethylene glycol, alkylpolyglycosides, N-alkylglucamine derivatives, etc.

Organic Inert Phase

The compositions may comprise at least one organic inert phase.

The term “inert” is understood to mean not causing a rapid destruction of the persulfates, i.e. not causing a decrease in the persulfate level of more than 50% in 24 hours at ambient temperature. Preferably, the organic inert phase is a fatty phase consisting of one or more fatty substances.

The term “fatty substance” is understood to mean an organic compound that is insoluble in water at ordinary temperature (25° C.) and at atmospheric pressure (760 mmHg) (solubility of less than 5%, preferably less than 1% and even more preferentially less than 0.1%).

The fatty substances have in their structure at least one hydrocarbon-based chain containing at least 6 carbon atoms or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane. The fatty substances do not contain any salified carboxylic acid groups.

In particular, the fatty substances are also not (poly)oxyalkylenated or (poly)glycerolated ethers.

Preferably, the composition comprises a liquid organic inert phase (liquid fatty phase), comprising oils as fatty substances. For the purposes of the present disclosure, the term “liquid phase” is understood to mean any phase that is capable of flowing at ambient temperature, generally between 15° C. and 40° C., and at atmospheric pressure, under the action of its own weight.

The organic inert liquid phase can in particular be chosen from the polydecenes of formula C₁₀ₙH₍₂₀ₙ₎₊₂ in which n ranges from 3 to 9 and preferably from 3 to 7, liquid fatty alcohols, esters of fatty alcohols or of fatty acids, sugar esters or diesters of C12-C24 fatty acids, cyclic ethers or cyclic esters, silicone oils, mineral oils or plant oils, or mixtures thereof.

The compounds of formula C₁₀ₙH₍₂₀ₙ₎₊₂ in which n ranges from 3 to 9 correspond to the name “polydecene” of the CTFA dictionary, 7th edition, 1997 of the Cosmetic, Toiletry and Fragrance Association, USA, and also to the same INCI name in the USA and in Europe. These are poly-1-decene hydrogenation products.

Among these compounds, those for which, in the formula, n ranges from 3 to 7 are preferred.

Examples that may be mentioned include the product sold under the name Silkflo® 366 NF Polydecene by the company Amoco Chemical, and those sold under the name Nexbase® 2002 FG, 2004 FG, 2006 FG and 2008 FG by the company Fortum.

As regards the esters of fatty alcohols or of fatty acids, examples that may be mentioned include:

esters of saturated, linear or branched C₃-C₆ lower monoalcohols with monofunctional C12-C24 fatty acids, these fatty acids possibly being linear or branched, saturated or unsaturated and chosen in particular from oleates, laurates, palmitates, myristates, behenates, cocoates, stearates, linoleates, linolenates, caprates and arachidonates, or mixtures thereof, and in particular oleo-palmitates, oleo-stea rates and palmito-stearates. Among these esters, it is more particularly preferred to use isopropyl palmitate, isopropyl myristate and octyldodecyl stearate,

esters of linear or branched C₃-C₈ monoalcohols with difunctional C₈-C₂₄ fatty acids, these fatty acids possibly being linear or branched, and saturated or unsaturated, for instance the isopropyl diester of sebacic acid, also known as diisopropyl sebacate,

esters of linear or branched C₃-C₈ monoalcohols with difunctional C₂-C₈ fatty acids, these fatty acids possibly being linear or branched, and saturated or unsaturated, for instance dioctyl adipate and dicaprylyl maleate,

the ester of a trifunctional acid, for instance triethyl citrate.

As regards the sugar esters and diesters of C12-C24 fatty acids, the term “sugar” is understood to mean compounds containing several alcohol functions, with or without an aldehyde or ketone function, and which comprise at least 4 carbon atoms. These sugars can be monosaccharides, oligosaccharides or polysaccharides.

As sugars that may be used, examples that may be mentioned include sucrose (or saccharose), glucose, galactose, ribose, fucose, maltose, fructose, mannose, arabinose, xylose and lactose, and derivatives thereof, in particular alkyl derivatives such as methyl derivatives, for instance methylglucose.

The esters of sugars and of fatty acids that may be used may be chosen in particular from the group comprising esters or mixtures of esters of sugars described above and of linear or branched, saturated or unsaturated C12-C24 fatty acids.

The esters may be chosen from mono-, di-, tri-, tetraesters and polyesters, and mixtures thereof.

These esters may be chosen, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferred to use monoesters and diesters and in particular sucrose, glucose or methyl-glucose mono- or dioleates, stearates, behenates, oleopalmitates, linoleates, linolenates and oleo-stearates.

Mention may be made, by way of example, of the product sold under the name Glucate® DO by Amerchol, which is a methylglucose dioleate.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% diester, triester and polyester;
the sucrose monodipalmitostearate sold by the company Goldschmidt under the name Tegosoft® PSE.

As regards the cyclic ethers and cyclic esters, γ-butyrolactone, dimethyl isosorbide and diisopropyl isosorbide are in particular suitable.

Silicone oils may also be used as inert organic liquid phase.

More particularly, the silicone oils that are suitable are liquid, non-volatile silicone fluids with a viscosity of less than or equal to 10 000 mPa·s at 25° C., the viscosity of the silicones being measured according to ASTM standard 445 Appendix C.

Silicone oils are defined in greater detail in Walter Noll's “Chemistry and Technology of Silicones” (1968)—Academic Press.

Among the silicone oils that may be used, mention may be made in particular of the silicone oils sold under the names DC-200 Fluid—5 mPa·s, DC-200 Fluid—20 mPa·s, DC-200 Fluid—350 mPa·s, DC-200 Fluid-1000 mPa·s and DC-200 Fluid—10 000 mPa·s by the company Dow Corning. Mineral oils may also be used as inert organic liquid phase, for instance liquid paraffin.

Plant oils may also be suitable for use, and in particular avocado oil, olive oil or liquid jojoba wax.

Preferably, the inert organic liquid phase is chosen from the group formed by polydecenes of formula C₁₀ₙH₍₂₀ₙ₎₊₂ in which n ranges from 3 to 9 and preferably from 3 to 7, and esters of fatty alcohols or of fatty acids, and mixtures thereof. According to one particular embodiment, the content of organic inert phase, which is preferably liquid, ranges from 0.1% to 30% by weight, preferably from 0.5% to 20% by weight and even more preferentially from 1% to 10% by weight relative to the weight of the composition n.

In one embodiment, a composition is anhydrous when it has a water content of less than 1% by weight and preferably less than 0.5% by weight relative to the total weight of the composition. In one embodiment, the composition is free of water.

The composition may also comprise various additives conventionally used in cosmetics and which may be present in the first and/or the second layer of the composition.

The composition may thus comprise lubricants, for instance polyol stearates or alkali metal or alkaline-earth metal stearates, pigments, colouring agents, additives such as urea, ammonium chloride, antioxidants, penetrants, sequestrants such as EDTA or EDDS, buffers, dispersants, film-forming agents, preservatives, opacifiers, vitamins, fragrances, anionic, non-ionic, amphoteric or zwitterionic polymers other than the rheology modifiers already mentioned, conditioning agents, for instance cationic polymers, ceramides and amino silicones.

In particular, the composition may comprise at least one colouring agent chosen from oxidation dye precursors, direct dyes or mixtures thereof, which will be detailed below. The colouring agent may be present in the first and/or in the second layer.

The oxidation dye precursors are generally chosen from oxidation bases, couplers, and mixtures thereof.

By way of example, the oxidation bases are chosen from para-phenylenediamines, bis(phenyl)alkylenediamines, para-aminophenols, ortho-aminophenols, heterocyclic bases, for instance pyridine derivatives, pyrimidine derivatives and pyrazole derivatives, and the addition salts thereof.

The composition may optionally comprise one or more couplers.

Mention may in particular be made, among these couplers, of meta-phenylenediamines, meta-aminophenols, meta-diphenols, naphthalene-based couplers and heterocyclic couplers, and also the addition salts thereof.

In general, the addition salts of the oxidation bases and couplers that can be used chosen from the addition salts with an acid such as the hydrochlorides, hydrobromides, sulfates, citrates, succinates, tartrates, lactates, tosylates, benzenesulfonates, phosphates and acetates.

According to one embodiment, the composition may comprise, besides the first and second layers, at least one layer comprising breakdown agents intended to accelerate the disintegration of the tablet, alkaline agents as mentioned above, and cosmetic active agents, and mixtures thereof. Breakdown agents that may in particular be mentioned include celluloses and cellulose derivatives, in particular hydroxyalkylcelluloses, amphiphilic polyurethanes, crosslinked polyacrylates, crosslinked polyvinylpyrrolidone, gums, such as guar gum, soybean polysaccharides, alginates, aluminium silicates and derivatives thereof, and silicas, in particular hydrophilic silicas, and mixtures thereof.

The present disclosure also relates to a process for bleaching keratin fibres, which consists in applying to the keratin fibres a bleaching composition which is in compressed form, comprising at least one first layer comprising at least one persulfate and at least one second layer comprising at least one hydrogen peroxide generator, in the presence of an aqueous composition.

The composition in compressed form is generally added to the aqueous composition just at the time of use, i.e. just before application to the keratin fibres. The step of dissolution of the bleaching composition in compressed form may take a few seconds to a few minutes, and may be performed with or without stirring.

The suitable medium for the aqueous composition generally consists of water or a mixture of water and at least one organic solvent to dissolve the compounds that are not sufficiently water-soluble. Examples of organic solvents that may be mentioned include Ci-C₄ lower alkanols, such as ethanol and isopropanol; polyols such as propylene glycol, glycerol, dipropylene glycol and polyol ethers, for instance 2-butoxyethanol, propylene glycol monomethyl ether, and also aromatic alcohols, for instance benzyl alcohol or phenoxyethanol, similar products and mixtures thereof.

The solvents may be present in proportions preferably of between 1% and 40% by weight and more preferably still between 5% and 30% by weight approximately relative to the total weight of the aqueous composition.

Preferably, the aqueous composition is constituted of water.

The aqueous composition may be in any form suitable to allow good dilution of the composition in compressed form, preferably in liquid form.

The composition may also contain various additives conventionally used in cosmetics, such as those described previously.

It may also comprise agents for controlling the release of oxygen, such as magnesium carbonate or oxide.

The additives and the oxygen-release control agents as defined previously may be present in an amount, for each of them, of between 0.01% and 40% by weight and preferably between 0.1% and 30% by weight relative to the total weight of the aqueous composition.

### Developer

In one embodiment, a developer includes at least one, more than one, or all of the following: water, mineral oil, hydrogen peroxide, cetearyl alcohol, steareth-20, PEG-4 rapeseedamide, glycerin, polyquaternium-6, hexadimethrine chloride, tocopherol pentasoidum pentetate, sodium stannate tetrasodium phyrophosphate, and phosphoric acid.

US20120325244, incorporated herein by reference, teaches developer compositions that are usable in the package 100 in one or both of the chambers 102, 104 or in the package 200.

The developer composition may be in the form of a powder, gel, liquid, foam, lotion, cream, mousse, and emulsion.

In one particular embodiment, the developer composition is aqueous or is in the form of an emulsion.

In another embodiment, the developer composition is substantially anhydrous. The term “substantially anhydrous” means that the developer composition is either completely free of water or contains no appreciable amount of water, for example, no more than 5% by weight, or no more than 2% by weight, or no more than 1% by weight, based on the weight of the developer composition. It should be noted that this refers for example to bound water, such as the water of crystallization of the salts or traces of water absorbed by the raw materials used in the preparation of the compositions according to the disclosure.

The developer composition can contain at least one solvent, chosen from water, organic solvents, and mixtures thereof.

When the developer composition is substantially anhydrous, the developer composition may comprise at least one solvent chosen from organic solvents. Suitable organic solvents for use in the developer composition include ethanol, isopropyl alcohol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The at least one solvent may, for example, be present in an amount ranging from about 0.5% to about 70% by weight, such as from about 2% to about 60% by weight, preferably from about 5% to about 50% by weight, relative to the total weight of the developer composition.

The pH of the developer composition can range from 2 to 12, such as from 6 to 11, and it may be adjusted to the desired value using acidifying/alkalizing agents that are well known in the art.

US20110209720, incorporated herein by reference, teaches developer compositions that are usable in the package 100 in one or both of the chambers 102, 104 or in the package 200.

In one embodiment, a devloper is formed from the combination of an anhydrous oxidizer composition containing at least one oxidizing agent.

The at least one oxidizing agent in the anhydrous oxidizer composition is selected from persulfates, perborates, percarbonates, their salts, and mixtures thereof.

Preferred persulfates are monopersulfates, their salts and mixtures thereof such as, for example, potassium persulfate, sodium persulfate, ammonium persulfate, as well as mixtures thereof.

The preferred oxidizing are potassium persulfate, sodium persulfate and mixtures thereof.

The term “anhydrous” means that the oxidizer composition is either completely free of water or contains no appreciable amount of water, preferably no more than 1% by weight, and more preferably no more than 0.5% by weight, based on the total weight of the anhydrous oxidizer composition.

According to a particularly preferred embodiment, the anhydrous oxidizer composition is totally anhydrous, that is to say it does not contain any water.

The anhydrous oxidizer composition can contain organic solvents, surfactants, silicones, and mixtures thereof.

Suitable organic solvents include ethanol, isopropyl alcohol, benzyl alcohol, phenyl ethyl alcohol, glycols and glycol ethers, such as ethylene glycol, propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The at least one organic solvent may, for example, be present in an amount ranging from 0.5% to 70% by weight, such as from 2% to 60% by weight, preferably from 5 to 50% by weight, relative to the total weight of the anhydrous oxidizer composition.

The anhydrous oxidizer composition may be in the form of a powder, gel, liquid, foam, lotion, cream, mousse, and emulsion.

In one embodiment, the oxidizer composition is in powder form.

In one embodiment, the oxidizer composition is in the form of a gel.

Suitable surfactants include:

(i) anionic surfactants such as, for example salts (such as alkaline salts, for example sodium salts, ammonium salts, amine salts, amino alcohol salts, and magnesium salts) of the following compounds: alkyl sulphates, alkyl ether sulphates, alkylamido ether sulphates, alkylaryl polyether sulphates, monoglyceride sulphates, alkyl sulphonates, alkyl Phosphates, alkylamide sulphonates, alkylaryl sulphonates, .alpha.-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates, and N-acyltaurates, wherein the alkyl or acyl radical of all of these various compounds may have from 12 to 20 carbon atoms, and the aryl radical may be chosen from phenyl and benzyl groups. Among the at least one anionic surfactant that may be used, mention may also be made of fatty acid salts such as the salts of oleic, ricinoleic, palmitic, and stearic acids; coconut oil acid; hydrogenated coconut oil acid; and acyl lactylates in which the acyl radical contains 8 to 20 carbon atoms. Use may also be made of at least one weakly anionic surfactant, such as alkyl-D-galactosiduronic acids and their salts, and polyoxyalkylenated carboxylic ether acids and their salts, such as those containing from 2 to 50 ethylene oxide groups. Anionic surfactants of the polyoxyalkylenated carboxylic ether acid or salt type may, for example, correspond to formula (1) below:

R₁—OC₂H₄)ₙ—OCH₂COOA (1)

in which:

R₁ is chosen from alkyl, alkylamido, and alkaryl groups, and n is chosen from integers and decimal numbers (average value) that may range from 2 to 24, such as from 3 to 10, wherein the alkyl radical has between 6 and 20 carbon atoms approximately, and the aryl radical may be a phenyl;

A is chosen from hydrogen, ammonium, Na, K, Li, Mg, monoethanolamine, and triethanolamine residues. Mixtures of compounds of formula (1) can also be used, for example mixtures in which the groups R₁ are different.

(ii) Nonionic Surfactants:

The at least one nonionic surfactant may be chosen from (as a non-limiting list) polyethoxylated, polypropoxylated, and polyglycerolated fatty alcohols; polyethoxylated, polypropoxylated, and polyglycerolated fatty .alpha.-diols; polyethoxylated, polypropoxylated, and polyglycerolated fatty alkylphenols; and polyethoxylated, polypropoxylated, and polyglycerolated fatty acids, all having a fatty chain containing, for example, 8 to 18 carbon atoms, it being possible for the number of ethylene oxide or propylene oxide groups to range, for example, from 2 to 50 and for the number of glycerol groups to range, for example, from 2 to 30. Mention may also be made of copolymers of ethylene oxide and of propylene oxide; condensates of ethylene oxide and of propylene oxide with fatty alcohols; polyethoxylated fatty amides, for example polyoethoxylated fatty amides having from 2 to 30 mol of ethylene oxide; polyglycerolated fatty amides having on average 1 to 5, such as 1.5 to 4, glycerol groups; oxyethylenated fatty acid esters of sorbitan having from 2 to 30 mol of ethylene oxide; fatty acid esters of sucrose; fatty acid esters of polyethylene glycol alkylpolyglycosides; N-alkylglucamine derivatives; amine oxides such as (C₁₀-C₁₄) alkylamineoxides; and N-acylaminopropylmorpholine oxides. The alkylpolyglycosides may also be mentioned as nonionic surfactants that are suitable in the context of the present disclosure.

(iii) Amphoteric or Zwitterionic Surfactants:

The at least one amphoteric or zwitterionic surfactant can be, for example (as a non-limiting list), aliphatic secondary and tertiary amine derivatives in which the aliphatic radical is a linear or branched chain containing 8 to 18 carbon atoms and containing at least one water-soluble anionic group (for example carboxylate, sulphonate, sulphate, phosphate and phosphonate groups); mention may also be made of (C₈-C₂₀)alkylbetaines, sulphobetaines, (C₈-C₂₀) alkylamido (C₁-C₆)alkylbetaines, and (C₈-C₂₀)alkylamido(C₁-C₆)alkylsulphobetaines.

Among the amine derivatives, mention may be made of the products sold under the name Miranol classified in the CTFA dictionary, 3rd edition, 1982, under the names Amphocarboxyglycinates and Amphocarboxypropionates, which correspond to the respective preferred structures (2) and (3):

R₂—CONHCH₂CH₂—N⁺(R₃)(R₄)(CH₂COO⁻ (2)

in which:

R₂ is chosen from alkyl radicals of an acid R₂—COOH present in hydrolysed coconut oil, heptyl radicals, nonyl radicals, and undecyl radicals, R₃ denotes a β-hydroxyethyl group, R₄ denotes a carboxymethyl group;

and R₂′—CONHCH₂CH₂—N(B)(D) (3)

in which:

B represents —CH₂CH₂OX′, D represents —(CH₂)_{z}—Y′, wherein z is chosen from 1 and 2,

X′ is chosen from —CH₂CH₂—COOH and hydrogen,

Y′ is chosen from —COOH and —CH₂—CHOH—SO₃H,

R₂′ is chosen from alkyl radicals, such as alkyl radicals of an acid R₂—COOH present in coconut oil or in hydrolysed linseed oil; C₇, C₉, C₁₁, C₁₃ alkyl radicals, C₁₇ alkyl radicals and its iso form; and unsaturated C₁₇ radicals.

(iv) Cationic Surfactants:

The at least one cationic surfactant may be chosen, for example, from: salts of optionally polyoxyalkylenated primary, secondary and tertiary fatty amines; quaternary ammonium salts such as tetra alkyl ammonium, alkylamidoalkyltrialkyl ammonium, trialkylbenzyl ammonium, trialkylhydroxyalkyl ammonium and alkylpyridinium chlorides and bromides; imidazoline derivatives; and cationic amine oxides.

In one embodiment, a developer composition contains the at least one oxidizing agent in an amount ranging from 1% to 80% by weight, preferably from 5% to 75% by weight, more preferably from 6% to 20% by weight, even more preferably from 6% to 10% by weight, based on the total weight of the developer composition.

### Additives

US 20120325244, incorporated herein by reference, teaches additives that are usable in the package 100 in one or both of the chambers 102, 104 or in the package 200.

As examples of additives that can be used, non-limiting mentions can be made of surfactants, antioxidants or reducing agents, penetrating agents, sequestering agents, perfumes, buffers, dispersants, conditioners, such as for example volatile or non-volatile, modified or unmodified silicones, film-forming agents, ceramides, preservatives, opacifiers, and antistatic agents.

While illustrative embodiments have been illustrated and described, it will be appreciated that various changes can be made therein without departing from the spirit and scope of the invention.

1. A package, comprising:
a water-dissolvable substrate forming the exterior of the package;
a pre-measured powdered hair bleach composition in at least one chamber of the package; and
a second composition in a second chamber of the package, the second chamber being separated by a barrier from the first chamber, and the second composition being the same or different from the powdered hair bleach composition.

2. The package of claim 1,
wherein the second composition is an additive composition configured to enhance a property of the powdered hair bleach.

3. The package of claim 1,
wherein the second composition is an anhydrous liquid composition.

4. A package, comprising:
a water-dissolvable substrate forming the exterior of the package; and
an anhydrous hair bleach developer liquid composition in a chamber of the package.

5. The package of claim 1, comprising:
wherein the second composition is an anhydrous liquid hair bleach composition.

6. A package, comprising:
a water-dissolvable substrate forming the exterior of the package; and
a pre-measured composition in at least one chamber of the package, wherein the water-dissolvable substrate includes hydrophilic polymers or a disintegrant.

7. The package of claim 4,
wherein the water-dissolvable substrate includes a release mechanism triggered by one of at least moisture and friction.

8. The package of claim 1,
wherein the water-dissolvable substrate interacts with an external agent to enhance a property of one or both compositions.

9. The package of claim 1,
wherein the water-dissolvable substrate is made from woven or non-woven fibers, wherein the fibers are impregnated with a hair bleach developer composition.

10. The package of claim 1,
wherein the water-dissolvable substrate is consumed in a reaction with water.

11. The package of claim 1, comprising:
a first and second water-dissolvable substrate forming the exterior of the package; and
a first pre-measured composition in a first chamber formed from the first water-dissolvable substrate; and
a second pre-measured composition in a second chamber formed from the second water-dissolvable substrate, wherein the first and second water-dissolvable substrates have different rates of dissolution.

12. The package of claim 1, comprising a synthetic or plant-derived dissolvable substrate and compositions.

13. A container, comprising:
a plurality of the packages of claim 1.
